SEQUENCE LISTING (37 sequences)

SEQ ID NO: 1 - DNA, 3924 bp, Homo sapiens
  source 1..3924: mol_type = other DNA
agtctcgagctctcgctggccttcgggtgtacgtgctccgggatcttcagcacccgcggccgccatcgccgtcgcttggcttcttctggactcatctgcgccacttgtccgcttcacactccgccgccatcatggtgaagctcgcgaaggcaggtaaaaatcaaggtgaccccaagaaaatggctcctcctccaaaggaggtagaagaagatagtgaagatgaggaaatgtcagaagatgaagaagatgatagcagtggagaagaggtcgtcatacctcagaagaaaggcaagaaggctgctgcaacctcagcaaagaaggtggtcgtttccccaacaaaaaaggttgcagttgccacaccagccaagaaagcagctgtcactccaggcaaaaaggcagcagcaacacctgccaagaagacagttacaccagccaaagcagttaccacacctggcaagaagggagccacaccaggcaaagcattggtagcaactcctggtaagaagggtgctgccatcccagccaagggggcaaagaatggcaagaatgccaagaaggaagacagtgatgaagaggaggatgatgacagtgaggaggatgaggaggatgacgaggacgaggatgaggatgaagatgaaattgaaccagcagcgatgaaagcagcagctgctgcccctgcctcagaggatgaggacgatgaggatgacgaagatgatgaggatgacgatgacgatgaggaagatgactctgaagaagaagctatggagactacaccagccaaaggaaagaaagctgcaaaagttgttcctgtgaaagccaagaacgtggctgaggatgaagatgaagaagaggatgatgaggacgaggatgacgacgacgacgaagatgatgaagatgatgatgatgaagatgatgaggaggaggaagaagaggaggaggaagagcctgtcaaagaagcacctggaaaacgaaagaaggaaatggccaaacagaaagcagctcctgaagccaagaaacagaaagtggaaggcacagaaccgactacggctttcaatctctttgttggaaacctaaactttaacaaatctgctcctgaattaaaaactggtatcagcgatgtttttgctaaaaatgatcttgctgttgtggatgtcagaattggtatgactaggaaatttggttatgtggattttgaatctgctgaagacctggagaaagcgttggaactcactggtttgaaagtctttggcaatgaaattaaactagagaaaccaaaaggaaaagacagtaagaaagagcgagatgcgagaacacttttggctaaaaatctcccttacaaagtcactcaggatgaattgaaagaagtgtttgaagatgctgcggagatcagattagtcagcaaggatgggaaaagtaaagggattgcttatattgaatttaagacagaagctgatgcagagaaaacctttgaagaaaagcagggaacagagatcgatgggcgatctatttccctgtactatactggagagaaaggtcaaaatcaagactatagaggtggaaagaatagcacttggagtggtgaatcaaaaactctggttttaagcaacctctcctacagtgcaacagaagaaactcttcaggaagtatttgagaaagcaacttttatcaaagtaccccagaaccaaaatggcaaatctaaagggtatgcatttatagagtttgcttcattcgaagacgctaaagaagctttaaattcctgtaataaaagggaaattgagggcagagcaatcaggctggagttgcaaggacccaggggatcacctaatgccagaagccagccatccaaaactctgtttgtcaaaggcctgtctgaggataccactgaagagacattaaaggagtcatttgacggctccgttcgggcaaggatagttactgaccgggaaactgggtcctccaaagggtttggttttgtagacttcaacagtgaggaggatgccaaagctgccaaggaggccatggaagacggtgaaattgatggaaataaagttaccttggactgggccaaacctaagggtgaaggtggcttcgggggtcgtggtggaggcagaggcggctttggaggacgaggtggtggtagaggaggccgaggaggatttggtggcagaggccggggaggctttggagggcgaggaggcttccgaggaggcagaggaggaggaggtgaccacaagccacaaggaaagaagacgaagtttgaatagcttctgtccctctgctttcccttttccatttgaaagaaaggactctggggtttttactgttacctgatcaatgacagagccttctgaggacattccaagacagtatacagtcctgtggtctccttggaaatccgtctagttaacatttcaagggcaataccgtgttggttttgactggatattcatataaactttttaaagagttgagtgatagagctaacccttatctgtaagttttgaatttatattgtttcatcccatgtacaaaaccattttttcctacaaatagtttgggttttgttgttgtttcttttttttgttttgtttttgttttttttttttttgcgttcgtggggttgtaaaagaaaagaaagcagaatgttttatcatggtttttgcttcagcggctttaggacaaattaaaagtcaactctggtgccagacgtgttacttcctaaagagtgtttcccctggaatgtcactggagagcatggcaaagccagctctgccacttgcttcacccatcccaatggaaatggcttagtgcgtgtttccagtatcccagccctaactaacttggttgaaatgctggtgaggggacctgctcctgcagccctggtgctgacttgaaggctgctgcagcttctcctacttttagcaggtctgaggattatgtcctgaagaccactctggaaagaggtgcaggaacagattagtcaggtttcctaggacaaggaagagcttcagggaagagcagtggctaactcctgtaatcccaacacttggggaggccgaggcaggcagatcaactgaggtcaggagttgaagaccagcctggccaacatggtgaaagcccatctctactaaaaatacaaaaattagctgggcatggtggtgtactcctgtagtcccagctactcaggaggctgaagcgggagagtcacgtgaacccgggaagcagagtgagctgagcacacactactatactccaggctgggtaacaaagcgagactcccatctcccaaaaagcagttctggaatagaactcacgctagatggatagaccagtggacactttggaaccttggggctggggaggaaactgcccatccagtaaacccccaaaaagccatttgttctgcactacgtatattgcttattctttctggtcttaagtacttgcctctcaacctccctttttagtaaaagacaaggccacgtgagaggcgggactatcaacattgtgatgaatttacttgaaacccagtgcccaaaatcaatgtaggtagccaagtccaaaaacctgttctagtccaactagtgaaatcaaactgtgatacttggataagcttagaaggaaacgtgaagaatacgtagctgctttgggtttactctggttcagttgggctgttgaaatcttaacatccttgggcttatcacctactgcttgtcagccctgttccatgtccaggggatgggggtggtgacaatccagttccaagaccctcatgctctagagaggaaggtggccagccagggttgtaactacgatgaaaaagcagtgggagggtctcctatgaggcaagcctaaggacaaaaaggaaggccttgcagcctgtattctggataaggaattaaaagctcagttaattgaagccca

SEQ ID NO: 2 - DNA, 22 bp, synthetic construct
  misc_feature 1..22: note = Amplification oligomer
  source 1..22: mol_type = other DNA
gacagctcagaggaggaggatg

SEQ ID NO: 3 - DNA, 56 bp, synthetic construct
  misc_feature 1..56: note = Amplification oligomer
  source 1..56: mol_type = other DNA
gtcgtcgacaaatacctgtgtgtgataggagagggatatcactcagcataatttaa

SEQ ID NO: 4 - DNA, 29 bp, synthetic construct
  misc_feature 1..29: note = Target-hybridizing sequence of SEQ ID NO:3
  source 1..29: mol_type = other DNA
ggagagggatatcactcagcataatttaa

SEQ ID NO: 5 - RNA, 19 nt, synthetic construct
  misc_feature 1..19: note = Nucleolin detection probe
  source 1..19: mol_type = other RNA
gaggatgaagatgaaattg

SEQ ID NO: 6 - RNA, 20 nt, synthetic construct
  misc_feature 1..20: note = Nucleolin detection probe
  source 1..20: mol_type = other RNA
ggatgaagatgaaattgaac

SEQ ID NO: 7 - RNA, 19 nt, synthetic construct
  misc_feature 1..19: note = Nucleolin detection probe
  source 1..19: mol_type = other RNA
gatgaagatgaaattgaac

SEQ ID NO: 8 - RNA, 18 nt, synthetic construct
  misc_feature 1..18: note = Tubulin beta-4B detection probe
  source 1..18: mol_type = other RNA
agcaaatgcttaatgtcc

SEQ ID NO: 9 - RNA, 19 nt, synthetic construct
  misc_feature 1..19: note = Nucleolin detection probe
  misc_feature 7^8: note = acridinium ester label
  source 1..19: mol_type = other RNA
  modified_base 1: mod_base = gm
  modified_base 2: mod_base = OTHER; note = 2'-O-methyladenosine
  modified_base 3..4: mod_base = gm
  modified_base 5: mod_base = OTHER; note = 2'-O-methyladenosine
  modified_base 6: mod_base = um
  modified_base 7: mod_base = gm
  modified_base 8..9: mod_base = OTHER; note = 2'-O-methyladenosine
  modified_base 10: mod_base = gm
  modified_base 11: mod_base = OTHER; note = 2'-O-methyladenosine
  modified_base 12: mod_base = um
  modified_base 13: mod_base = gm
  modified_base 14..16: mod_base = OTHER; note = 2'-O-methyladenosine
  modified_base 17..18: mod_base = um
gaggatgaagatgaaattg

SEQ ID NO: 10 - RNA, 19 nt, synthetic construct
  misc_feature 1..19: note = Nucleolin detection probe
  misc_feature 8^9: note = acridinium ester label
  source 1..19: mol_type = other RNA
  modified_base 1: mod_base = gm
  modified_base 2: mod_base = OTHER; note = 2'-O-methyladenosine
  modified_base 3..4: mod_base = gm
  modified_base 5: mod_base = OTHER; note = 2'-O-methyladenosine
  modified_base 6: mod_base = um
  modified_base 7: mod_base = gm
  modified_base 8..9: mod_base = OTHER; note = 2'-O-methyladenosine
  modified_base 10: mod_base = gm
  modified_base 11: mod_base = OTHER; note = 2'-O-methyladenosine
  modified_base 12: mod_base = um
  modified_base 13: mod_base = gm
  modified_base 14..16: mod_base = OTHER; note = 2'-O-methyladenosine
  modified_base 17..18: mod_base = um
gaggatgaagatgaaattg

SEQ ID NO: 11 - RNA, 19 nt, synthetic construct
  misc_feature 1..19: note = Nucleolin detection probe
  misc_feature 10^11: note = acridinium ester label
  source 1..19: mol_type = other RNA
  modified_base 1: mod_base = gm
  modified_base 2: mod_base = OTHER; note = 2'-O-mehtyladenosine
  modified_base 3..4: mod_base = gm
  modified_base 5: mod_base = OTHER; note = 2'-O-methyladenosine
  modified_base 6: mod_base = um
  modified_base 7: mod_base = gm
  modified_base 8..9: mod_base = OTHER; note = 2'-O-methyladenosine
  modified_base 10: mod_base = gm
  modified_base 11: mod_base = OTHER; note = 2'-O-methyladenosine
  modified_base 12: mod_base = um
  modified_base 13: mod_base = gm
  modified_base 14..16: mod_base = OTHER; note = 2'-O-methyladenosine
  modified_base 17..18: mod_base = um
gaggatgaagatgaaattg

SEQ ID NO: 12 - RNA, 20 nt, synthetic construct
  misc_feature 1..20: note = Nucleolin detection probe
  misc_feature 8^9: note = acridinium ester label
  source 1..20: mol_type = other RNA
  modified_base 1..2: mod_base = gm
  modified_base 3: mod_base = OTHER; note = 2'-O-methyladenosine
  modified_base 4: mod_base = um
  modified_base 5: mod_base = gm
  modified_base 6..7: mod_base = OTHER; note = 2'-O-methyladenosine
  modified_base 8: mod_base = gm
  modified_base 9: mod_base = OTHER; note = 2'-O-methyladenosine
  modified_base 10: mod_base = um
  modified_base 11: mod_base = gm
  modified_base 12..14: mod_base = OTHER; note = 2'-O-methyladenosine
  modified_base 15..16: mod_base = um
  modified_base 17: mod_base = gm
  modified_base 18..19: mod_base = OTHER; note = 2'-O-methyladenosine
ggatgaagatgaaattgaac

SEQ ID NO: 13 - RNA, 20 nt, synthetic construct
  misc_feature 1..20: note = Nucleolin detection probe
  misc_feature 9^10: note = acridinium ester label
  source 1..20: mol_type = other RNA
  modified_base 1..2: mod_base = gm
  modified_base 3: mod_base = OTHER; note = 2'-O-methyladenosine
  modified_base 4: mod_base = um
  modified_base 5: mod_base = gm
  modified_base 6..7: mod_base = OTHER; note = 2'-O-methyladenosine
  modified_base 8: mod_base = gm
  modified_base 9: mod_base = OTHER; note = 2'-O-methyladenosine
  modified_base 10: mod_base = um
  modified_base 11: mod_base = gm
  modified_base 12..14: mod_base = OTHER; note = 2'-O-methyladenosine
  modified_base 15..16: mod_base = um
  modified_base 17: mod_base = gm
  modified_base 18..19: mod_base = OTHER; note = 2'-O-methyladenosine
ggatgaagatgaaattgaac

SEQ ID NO: 14 - RNA, 20 nt, synthetic construct
  misc_feature 1..20: note = Nucleolin detection probe
  misc_feature 10^11: note = acridinium ester label
  source 1..20: mol_type = other RNA
  modified_base 1..2: mod_base = gm
  modified_base 3: mod_base = OTHER; note = 2'-O-methyladenosine
  modified_base 4: mod_base = um
  modified_base 5: mod_base = gm
  modified_base 6..7: mod_base = OTHER; note = 2'-O-methyladenosine
  modified_base 8: mod_base = gm
  modified_base 9: mod_base = OTHER; note = 2'-O-methyladenosine
  modified_base 10: mod_base = um
  modified_base 11: mod_base = gm
  modified_base 12..14: mod_base = OTHER; note = 2'-O-methyladenosine
  modified_base 15..16: mod_base = um
  modified_base 17: mod_base = gm
  modified_base 18..19: mod_base = OTHER; note = 2'-O-methyladenosine
ggatgaagatgaaattgaac

SEQ ID NO: 15 - RNA, 19 nt, synthetic construct
  misc_feature 1..19: note = Nucleolin detection probe
  misc_feature 7^8: note = acridinium ester label
  source 1..19: mol_type = other RNA
  modified_base 1: mod_base = gm
  modified_base 2: mod_base = OTHER; note = 2'-O-methyladenosine
  modified_base 3: mod_base = um
  modified_base 4: mod_base = gm
  modified_base 5..6: mod_base = OTHER; note = 2'-O-methyladenosine
  modified_base 7: mod_base = gm
  modified_base 8: mod_base = OTHER; note = 2'-O-methyladenosine
  modified_base 9: mod_base = um
  modified_base 10: mod_base = gm
  modified_base 11..13: mod_base = OTHER; note = 2'-O-methyladenosine
  modified_base 14..15: mod_base = um
  modified_base 16: mod_base = gm
  modified_base 17..18: mod_base = OTHER; note = 2'-O-methyladenosine
gatgaagatgaaattgaac

SEQ ID NO: 16 - RNA, 19 nt, synthetic construct
  misc_feature 1..19: note = Nucleolin detection probe
  misc_feature 9^10: note = acridinium ester label
  source 1..19: mol_type = other RNA
  modified_base 1: mod_base = gm
  modified_base 2: mod_base = OTHER; note = 2'-O-methyladenosine
  modified_base 3: mod_base = um
  modified_base 4: mod_base = gm
  modified_base 5..6: mod_base = OTHER; note = 2'-O-methyladenosine
  modified_base 7: mod_base = gm
  modified_base 8: mod_base = OTHER; note = 2'-O-methyladenosine
  modified_base 9: mod_base = um
  modified_base 10: mod_base = gm
  modified_base 11..13: mod_base = OTHER; note = 2'-O-methyladenosine
  modified_base 14..15: mod_base = um
  modified_base 16: mod_base = gm
  modified_base 17..18: mod_base = OTHER; note = 2'-O-methyladenosine
gatgaagatgaaattgaac

SEQ ID NO: 17 - RNA, 19 nt, synthetic construct
  misc_feature 1..19: note = Nucleolin detection probe
  misc_feature 10^11: note = acridinium ester label
  source 1..19: mol_type = other RNA
  modified_base 1: mod_base = gm
  modified_base 2: mod_base = OTHER; note = 2'-O-methyladenosine
  modified_base 3: mod_base = um
  modified_base 4: mod_base = gm
  modified_base 5..6: mod_base = OTHER; note = 2'-O-methyladenosine
  modified_base 7: mod_base = gm
  modified_base 8: mod_base = OTHER; note = 2'-O-methyladenosine
  modified_base 9: mod_base = um
  modified_base 10: mod_base = gm
  modified_base 11..13: mod_base = OTHER; note = 2'-O-methyladenosine
  modified_base 14..15: mod_base = um
  modified_base 16: mod_base = gm
  modified_base 17..18: mod_base = OTHER; note = 2'-O-methyladenosine
gatgaagatgaaattgaac

SEQ ID NO: 18 - RNA, 18 nt, synthetic construct
  misc_feature 1..18: note = Tubulin beta-4B detection probe
  misc_feature 7^8: note = acridinium ester label
  source 1..18: mol_type = other RNA
  modified_base 1: mod_base = OTHER; note = 2'-O-methyladenosine
  modified_base 2: mod_base = gm
  modified_base 3: mod_base = cm
  modified_base 4..6: mod_base = OTHER; note = 2'-O-methyladenosine
  modified_base 7: mod_base = um
  modified_base 8: mod_base = gm
  modified_base 9: mod_base = cm
  modified_base 10..11: mod_base = um
  modified_base 12..13: mod_base = OTHER; note = 2'-O-methyladenosine
  modified_base 14: mod_base = um
  modified_base 15: mod_base = gm
  modified_base 16: mod_base = um
  modified_base 17: mod_base = cm
agcaaatgcttaatgtcc

SEQ ID NO: 19 - RNA, 18 nt, synthetic construct
  misc_feature 1..18: note = Tubulin beta-4B detection probe
  misc_feature 8^9: note = acridinium ester label
  source 1..18: mol_type = other RNA
  modified_base 1: mod_base = OTHER; note = 2'-O-methyladenosine
  modified_base 2: mod_base = gm
  modified_base 3: mod_base = cm
  modified_base 4..6: mod_base = OTHER; note = 2'-O-methyladenosine
  modified_base 7: mod_base = um
  modified_base 8: mod_base = gm
  modified_base 9: mod_base = cm
  modified_base 10..11: mod_base = um
  modified_base 12..13: mod_base = OTHER; note = 2'-O-methyladenosine
  modified_base 14: mod_base = um
  modified_base 15: mod_base = gm
  modified_base 16: mod_base = um
  modified_base 17: mod_base = cm
agcaaatgcttaatgtcc

SEQ ID NO: 20 - RNA, 18 nt, synthetic construct
  misc_feature 1..18: note = Tubulin beta-4B detection probe
  misc_feature 10^11: note = acridinium ester label
  source 1..18: mol_type = other RNA
  modified_base 1: mod_base = OTHER; note = 2'-O-methyladenosine
  modified_base 2: mod_base = gm
  modified_base 3: mod_base = cm
  modified_base 4..6: mod_base = OTHER; note = 2'-O-methyladenosine
  modified_base 7: mod_base = um
  modified_base 8: mod_base = gm
  modified_base 9: mod_base = cm
  modified_base 10..11: mod_base = um
  modified_base 12..13: mod_base = OTHER; note = 2'-O-methyladenosine
  modified_base 14: mod_base = um
  modified_base 15: mod_base = gm
  modified_base 16: mod_base = um
  modified_base 17: mod_base = cm
agcaaatgcttaatgtcc

SEQ ID NO: 21 - RNA, 18 nt, synthetic construct
  misc_feature 1..18: note = Tubulin beta-4B detection probe
  misc_feature 11^12: note = acridinium ester label
  source 1..18: mol_type = other RNA
  modified_base 1: mod_base = OTHER; note = 2'-O-methyladenosine
  modified_base 2: mod_base = gm
  modified_base 3: mod_base = cm
  modified_base 4..6: mod_base = OTHER; note = 2'-O-methyladenosine
  modified_base 7: mod_base = um
  modified_base 8: mod_base = gm
  modified_base 9: mod_base = cm
  modified_base 10..11: mod_base = um
  modified_base 12..13: mod_base = OTHER; note = 2'-O-methyladenosine
  modified_base 14: mod_base = um
  modified_base 15: mod_base = gm
  modified_base 16: mod_base = um
  modified_base 17: mod_base = cm
agcaaatgcttaatgtcc

SEQ ID NO: 22 - RNA, 23 nt, synthetic construct
  misc_feature 1..23: note = HPVA1 detection probe
  source 1..23: mol_type = other RNA
cagctggacaagcagaaccggac

SEQ ID NO: 23 - RNA, 21 nt, synthetic construct
  misc_feature 1..21: note = HPVA2 detection probe
  source 1..21: mol_type = other RNA
ggccagatggacaagcacaac

SEQ ID NO: 24 - RNA, 25 nt, synthetic construct
  misc_feature 1..25: note = HPV18 detection probe
  source 1..25: mol_type = other RNA
gtagtagaaagctcagcagacgacc

SEQ ID NO: 25 - RNA, 21 nt, synthetic construct
  misc_feature 1..21: note = HPV45 detection probe
  source 1..21: mol_type = other RNA
gtagagagctcggcagaggac

SEQ ID NO: 26 - RNA, 23 nt, synthetic construct
  misc_feature 1..23: note = HPV59 detection probe
  source 1..23: mol_type = other RNA
gtagtagaaacctcgcaagacgg

SEQ ID NO: 27 - RNA, 21 nt, synthetic construct
  misc_feature 1..21: note = HPVC2 detection probe
  source 1..21: mol_type = other RNA
ccgaccatgcagttaatcacc

SEQ ID NO: 28 - RNA, 21 nt, synthetic construct
  misc_feature 1..21: note = HPV51 detection probe
  source 1..21: mol_type = other RNA
gcgtgaccagctaccagaaag

SEQ ID NO: 29 - RNA, 20 nt, synthetic construct
  misc_feature 1..20: note = HPVD detection probe
  source 1..20: mol_type = other RNA
cacgtaccttgttgtgagtg

SEQ ID NO: 30 - DNA, 66 bp, Homo sapiens
  source 1..66: mol_type = other DNA
  misc_feature 1..66: note = Nucleolin amplification oligomer binding region
gacagtgatgaagaggaggatgatgacagtgaggaggatgaggaggatgacgaggacgaggatgag

SEQ ID NO: 31 - DNA, 32 bp, Homo sapiens
  source 1..32: mol_type = other DNA
  misc_feature 1..32: note = Nucleolin amplification oligomer binding region
cagcagcgatgaaagcagcagctgctgcccct

SEQ ID NO: 32 - DNA, 117 bp, Homo sapiens
  source 1..117: mol_type = other DNA
  misc_feature 1..117: note = Nucleolin target region
gacagtgatgaagaggaggatgatgacagtgaggaggatgaggaggatgacgaggacgaggatgaggatgaagatgaaattgaaccagcagcgatgaaagcagcagctgctgcccct

SEQ ID NO: 33 - RNA, 39 nt, synthetic construct
  source 1..39: mol_type = other RNA
gttcaatttcatcttcatcctcatcctcgtcctcgtcat

SEQ ID NO: 34 - RNA, 28 nt, synthetic construct
  source 1..28: mol_type = other RNA
tttttgttttggacattaagcatttgct

SEQ ID NO: 35 - skipped

SEQ ID NO: 36 - RNA, 39 nt, synthetic construct; identical to SEQ ID NO: 33
  source 1..39: mol_type = other RNA
  modified_base 1: mod_base = gm
  modified_base 2..3: mod_base = um
  modified_base 4: mod_base = cm
  modified_base 5..6: mod_base = OTHER; note = 2'-O-methyladenosine
  modified_base 7..9: mod_base = um
  modified_base 10: mod_base = cm
  modified_base 11: mod_base = OTHER; note = 2'-O-methyladenosine
  modified_base 12: mod_base = um
  modified_base 13: mod_base = cm
  modified_base 14..15: mod_base = um
  modified_base 16: mod_base = cm
  modified_base 17: mod_base = OTHER; note = 2'-O-methyladenosine
  modified_base 18: mod_base = um
  modified_base 19..20: mod_base = cm
  modified_base 21: mod_base = um
  modified_base 22: mod_base = cm
  modified_base 23: mod_base = OTHER; note = 2'-O-methyladenosine
  modified_base 24: mod_base = um
  modified_base 25..26: mod_base = cm
  modified_base 27: mod_base = um
  modified_base 28: mod_base = cm
  modified_base 29: mod_base = gm
  modified_base 30: mod_base = um
  modified_base 31..32: mod_base = cm
  modified_base 33: mod_base = um
  modified_base 34: mod_base = cm
  modified_base 35: mod_base = gm
  modified_base 36: mod_base = um
  modified_base 37: mod_base = cm
  modified_base 38: mod_base = OTHER; note = 2'-O-methyladenosine

SEQ ID NO: 37 - RNA, 28 nt, synthetic construct
  source 1..28: mol_type = other RNA
  modified_base 1..5: mod_base = um
  modified_base 6: mod_base = gm
  modified_base 7..10: mod_base = um
  modified_base 11..12: mod_base = gm
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16..17: mod_base = um
  modified_base 18..19: mod_base = OTHER; note = 2'-O-methyladenosine
  modified_base 20: mod_base = gm
  modified_base 21: mod_base = cm
  modified_base 22: mod_base = OTHER; note = 2'-O-methyladenosine
  modified_base 23..25: mod_base = um
  modified_base 26: mod_base = gm
  modified_base 27: mod_base = cm
tttttgttttggacattaagcatttgct

(19) United States
(12) Patent Application Publication
(10) Pub. No.: US 2026/0132477 A1
(43) Pub. Date: May 14, 2026

(54) CELLULARITY CONTROL FOR NUCLEIC ACID ASSAYS

(71) Applicant: GEN-PROBE INCORPORATED, San Diego, CA (US)

(72) Inventors: Damon K. GETMAN, Poway, CA (US); Analee R. WILLIAMS, San Diego, CA (US)

(73) Assignee: GEN-PROBE INCORPORATED, San Diego, CA (US)

(21) Appl. No.: 19/120,025

(22) PCT Filed: Oct. 12, 2023
(86) PCT No.: PCT/US2023/076676

§ 371 Date: Apr. 10, 2025

Related U.S. Application Data
(60) Provisional application No. 63/379,339, filed on Oct. 13, 2022.

(51) Int. Cl. C12Q 1/70; C12Q 1/6816; C12Q 1/6848

(52) U.S. Cl. CPC C12Q 1/708; C12Q 1/6816; C12Q 1/6848; C12Q 2600/16

(57) ABSTRACT

Disclosed are compositions and methods for performing nucleic-acid-based assays to determine the presence or absence of a target nucleic acid in a biological sample. The disclosed methods include the use of one or more oligomers that specifically hybridize to a nucleolin nucleic acid to assess the cellularity of the biological sample. Also disclosed are related composition and methods for assessing the cellularity of a biological sample for use in a molecular diagnostic assay.

### CROSS-REFERENCE TO RELATED APPLICATIONS

This application claims the benefit of U.S. Provisional Application No. 63/379,339, filed Oct. 13, 2022, which is incorporated by reference herein in its entirety.

### REFERENCE TO SEQUENCE LISTING

The instant application contains a Sequence Listing which has been submitted electronically in XML format and is hereby incorporated by reference in its entirety. Said XML Copy, created on Sep. 27, 2023, is named “4340-P22WO_Seq_Listing” and is 144,023 bytes in size.

### BACKGROUND

Nucleic-acid-based assays, including nucleic acid amplification methods, are widely used in many bioscience applications. Clinical diagnostic methods targeting nucleic acids from biological samples typically employ an internal control to ensure that the assay method is performing as intended. Such methods also typically rely on collection of a biological specimen containing a sufficient number of eukaryotic (e.g., human) cells that may contain a target such as, for example, a pathogenic organism. Internal controls, however, often rely on an exogenous target (i.e., a target not normally present in the tissue or cells being tested), and thus often fail to provide information on whether a sample contains a sufficient number of cells to proceed with an assay for the intended target.

### SUMMARY

In one aspect, the present invention provides a method for performing an amplification assay to determine the presence or absence of a target nucleic acid in a biological sample. The method generally includes the following steps: (a) providing a biological sample suspected of containing eukaryotic cells that may comprise the target nucleic acid; (b) contacting the sample with at least two control amplification oligomers and at least two target-specific amplification oligomers, wherein the at least two control amplification oligomers are capable of amplifying a target region of a nucleolin control nucleic acid, and wherein the at least two target-specific amplification oligomers are capable of amplifying a target region of the target nucleic acid; (c) performing an in vitro nucleic acid amplification reaction, wherein any nucleolin control nucleic acid present in the sample is used as a template for generating a control amplification product, and wherein any target nucleic acid present in the sample is used as a template for generating a target amplification product; and (d) detecting the presence or absence of each of the control amplification product and the target amplification product, wherein if the control amplification product is present, then the presence of the eukaryotic cells in the sample is assured and the presence or absence of the target amplification product determines, respectively, the presence or absence of the target nucleic acid in the sample.

In another aspect, the present invention provides a composition or kit for performing an amplification assay to determine the presence or absence of a target nucleic acid in a sample. The composition or kit generally includes at least two control amplification oligomers and at least two target-specific amplification oligomers, wherein the at least two control amplification oligomers are capable of amplifying a target region of a nucleolin control nucleic acid, and wherein the at least two target-specific amplification oligomers are capable of amplifying a target region of the target nucleic acid.

In another aspect, the present invention provides a composition or kit for assessing cellularity of a biological sample. The composition or kit generally includes (i) at least two amplification oligomers capable of amplifying a target region of a human nucleolin nucleic acid corresponding to a region of SEQ ID NO:1 from about nucleotide position 585 to about nucleotide position 677, and (ii) at least one detection probe oligomer configured to specifically hybridize to a nucleolin amplification product amplifiable by the at least two amplification oligomers.

In yet another aspect, the present invention provides a detection probe oligomer comprising a nucleolin-specific detection probe target-hybridizing sequence that is from about 15 to about 35 nucleotides in length and is configured to hybridize to a target sequence contained within a target region of human nucleolin nucleic acid corresponding to a region of SEQ ID NO:1 from about nucleotide position 585 to about nucleotide position 677.

In still another aspect, the present invention provides a method for assessing cellularity of a biological sample for use in a molecular diagnostic assay. The method generally includes the following steps: (a) providing a biological sample suspected of containing human cells; (b) contacting the sample with at least two amplification oligomers capable of amplifying a target region of a human nucleolin nucleic acid corresponding to a region of SEQ ID NO:1 from about nucleotide position 585 to about nucleotide position 677; (c) performing an in vitro nucleic acid amplification reaction, wherein any nucleolin nucleic acid present in the sample is used as a template for generating an amplification product; and (d) detecting the presence or absence of the amplification product, wherein the detecting step comprises contacting the in vitro nucleic acid amplification reaction with at least one detection probe oligomer configured to specifically hybridize to the amplification product under conditions whereby the presence or absence of the amplification product is determined, and wherein the presence of the amplification product indicates that the cellularity of the sample is adequate for molecular diagnostic testing.

Representative embodiments of these aspects are further set forth below.

### EMBODIMENTS

Embodiment 1. A method for performing an amplification assay to determine the presence or absence of a target nucleic acid in a biological sample, the method comprising: (a) providing a biological sample suspected of containing eukaryotic cells that may comprise the target nucleic acid; (b) contacting the sample with at least two control amplification oligomers and at least two target-specific amplification oligomers, wherein the at least two control amplification oligomers are capable of amplifying a target region of a nucleolin control nucleic acid, and wherein the at least two target-specific amplification oligomers are capable of amplifying a target region of the target nucleic acid; (c) performing an in vitro nucleic acid amplification reaction, wherein any nucleolin control nucleic acid present in the sample is used as a template for generating a control amplification product, and wherein any target nucleic acid present in the sample is used as a template for generating a target amplification product; and (d) detecting the presence or absence of each of the control amplification product and the target amplification product, wherein if the control amplification product is present, then the presence of the eukaryotic cells in the sample is assured and the presence or absence of the target amplification product determines, respectively, the presence or absence of the target nucleic acid in the sample.

Embodiment 2. The method of Embodiment 1, wherein the eukaryotic cells are human cells.

Embodiment 3. The method of Embodiment 2, wherein the at least two control amplification oligomers are capable of amplifying a target region of human nucleolin control nucleic acid corresponding to a region of SEQ ID NO:1 from about nucleotide position 585 to about nucleotide position 677.

Embodiment 4. The method of Embodiment 3, wherein the at least two control amplification oligomers comprise (i) a first control amplification oligomer comprising a first target-hybridizing sequence that targets a sequence contained with the region of SEQ ID NO:1 from nucleotide position 561 to nucleotide position 627, and/or (ii) a second control amplification oligomer comprising a second target-hybridizing sequence that targets a sequence contained with the region of SEQ ID NO:1 from nucleotide position 646 to nucleotide position 677.

Embodiment 5. The method of Embodiment 3a, wherein (i) the first control amplification oligomer comprises a first target-hybridizing sequence as shown in SEQ ID NO:2, and/or (ii) the second control amplification oligomer comprises a second target-hybridizing sequence as shown in SEQ ID NO:4.

Embodiment 6. The method of any one of Embodiments 2 to 5, wherein the sample is a tissue swab.

Embodiment 7. The method of Embodiment 6, wherein the tissue swab is a cervical swab, a vaginal swab, a nasal swab, a buccal swab, a throat swab, an anal swab, a urethral/penile swab, or a swab from an open wound/sore/laceration.

Embodiment 8. The method of any one of Embodiments 1 to 7, wherein the target nucleic acid is an RNA.

Embodiment 9. The method of any one of Embodiments 1 to 8, wherein the target nucleic acid is a pathogen-derived nucleic acid.

Embodiment 10. The method of Embodiment 9, wherein the pathogen-derived nucleic acid is a viral nucleic acid.

Embodiment 11. The method of Embodiment 10, wherein the viral nucleic acid is a human papillomavirus (HPV) nucleic acid.

Embodiment 12. The method of Embodiment 11, wherein the HPV nucleic acid is selected from the group consisting of HPV types 16 and 59.

Embodiment 13. The method of Embodiment 11, wherein the amplification assay is a multiplex amplification assay comprising detection of two or more types of HPV.

Embodiment 14. The method of Embodiment 13, wherein the two or more types of HPV comprise two or more of HPV types 16, 18, 31, 33, 35, 39, 45, 51, 52, 56, 58, 59, 66, and 68.

Embodiment 15. The method of Embodiment 14, wherein the two or more types of HPV comprise each of HPV type 16 (HPV16) and HPV type 59 (HPV59).

Embodiment 16. The method of Embodiment 15, wherein the at least two target-specific amplification oligomers comprise (i) an HPV16-specific amplification oligomer comprising a target-hybridizing sequence as shown in SEQ ID NO:2, and (ii) an HPV59-specific amplification oligomer comprising a target-hybridizing sequence as shown in SEQ ID NO:4.

Embodiment 17. The method of Embodiment 14, wherein the two or more types of HPV comprise each of HPV types 16, 18, 31, 33, 35, 39, 45, 51, 52, 56, 58, 59, 66, and 68.

Embodiment 18. The method of any one of Embodiments 1 to 17, wherein generating and detecting the control amplification product as defined in steps (b)-(d) is performed in a multiplex format with generating and detecting the target amplification product.

Embodiment 19. The method of any one of Embodiments 1 to 18, wherein the detecting step (d) comprises contacting the in vitro nucleic acid amplification reaction with (i) at least one control detection probe oligomer configured to specifically hybridize to the control amplification product under conditions whereby the presence or absence of the control amplification product is determined, and (ii) at least one target-specific detection probe oligomer configured to specifically hybridize to the target amplification product under conditions whereby the presence or absence of the target amplification product is determined, thereby detecting the presence or absence of each of the control amplification product and the target amplification product.

Embodiment 20. The method of any one of Embodiments 3 to 5, wherein the detecting step (d) comprises contacting the in vitro nucleic acid amplification reaction with (i) at least one control detection probe oligomer configured to specifically hybridize to the control amplification product under conditions whereby the presence or absence of the control amplification product is determined, and (ii) at least one target-specific detection probe oligomer configured to specifically hybridize to the target amplification product under conditions whereby the presence or absence of the target amplification product is determined, thereby detecting the presence or absence of each of the control amplification product and the target amplification product.

Embodiment 21. The method of Embodiment 20, wherein the at least one control detection probe oligomer comprises a target-hybridizing sequence selected from the group consisting of SEQ ID NO:5, SEQ ID NO:6, and SEQ ID NO:7.

Embodiment 22. The method of Embodiment 21, wherein the at least one control detection probe oligomer comprises a target hybridizing sequence as shown in SEQ ID NO:6.

Embodiment 23. The method of Embodiment 19, wherein each of the at least one control detection probe oligomer and the at least one target-specific detection probe oligomer comprises a detectable label.

Embodiment 24. The method of Embodiment 23, wherein the detectable label is a chemiluminescent or fluorescent label.

Embodiment 25. The method of Embodiment 24, wherein the detectable label is a chemiluminescent label.

Embodiment 26. The method of Embodiment 25, wherein the chemiluminescent label is an acridinium ester (AE) compound.

Embodiment 27. The method of Embodiment 24, wherein the detectable label is a fluorescent label and each of the at least one control detection probe oligomer and the at least one target-specific detection probe oligomer further comprises a non-fluorescent quencher.

Embodiment 28. The method of Embodiment 20, wherein each of the at least one control detection probe oligomer and the at least one target-specific detection probe oligomer comprises a detectable label.

Embodiment 29. The method of Embodiment 28, wherein the detectable label is a chemiluminescent or fluorescent label.

Embodiment 30. The method of Embodiment 29, wherein the detectable label is a chemiluminescent label.

Embodiment 31. The method of Embodiment 30, wherein the chemiluminescent label is an acridinium ester (AE) compound.

Embodiment 32. The method of Embodiment 31, wherein the at least one control detection probe oligomer comprises a target-hybridizing sequence selected from the group consisting of SEQ ID NO:9, SEQ ID NO:10, SEQ ID NO: 11, SEQ ID NO:12, SEQ ID NO:13, SEQ ID NO:14, SEQ ID NO:15, SEQ ID NO:16, and SEQ ID NO:17.

Embodiment 33. The method of Embodiment 32, wherein the at least one control detection probe oligomer comprises a target-hybridizing sequence selected from the group consisting of SEQ ID NO:10, SEQ ID NO:14, and SEQ ID NO:16.

Embodiment 34. The method of Embodiment 32, wherein the at least one control detection probe oligomer comprises a target-hybridizing sequence as shown in SEQ ID NO:14.

Embodiment 35. The method of Embodiment 29, wherein the detectable label is a fluorescent label and each of the at least one control detection probe oligomer and the at least one target-specific detection probe oligomer further comprises a non-fluorescent quencher.

Embodiment 36. The method of any one of Embodiments 1 to 35, wherein the in vitro nucleic acid amplification reaction is an isothermal amplification reaction.

Embodiment 37. The method of Embodiment 36, wherein the isothermal amplification reaction is a transcription-mediated amplification reaction.

Embodiment 38. A composition or kit for performing an amplification assay to determine the presence or absence of a target nucleic acid in a sample, the composition or kit comprising: at least two control amplification oligomers and at least two target-specific amplification oligomers, wherein the at least two control amplification oligomers are capable of amplifying a target region of a nucleolin control nucleic acid, and wherein the at least two target-specific amplification oligomers are capable of amplifying a target region of the target nucleic acid.

Embodiment 39. The composition or kit of Embodiment 38, wherein the at least two control amplification oligomers are capable of amplifying a target region of human nucleolin control nucleic acid corresponding to a region of SEQ ID NO:1 from about nucleotide position 585 to about nucleotide position 677.

Embodiment 40. The composition or kit of Embodiment 39, wherein the at least two control amplification oligomers comprise (i) a first control amplification oligomer comprising a first target-hybridizing sequence that targets a sequence contained with the region of SEQ ID NO:1 from nucleotide position 561 to nucleotide position 627, and/or (ii) a second control amplification oligomer comprising a second target-hybridizing sequence that targets a sequence contained with the region of SEQ ID NO:1 from nucleotide position 646 to nucleotide position 677.

Embodiment 41. The composition or kit of Embodiment 40, wherein (i) the first control amplification oligomer comprises a first target-hybridizing sequence as shown in SEQ ID NO:2, and/or (ii) the second control amplification oligomer comprises a second target-hybridizing sequence as shown in SEQ ID NO:4.

Embodiment 42. The composition or kit of any one of Embodiments 38 to 41, wherein the target nucleic acid is an RNA.

Embodiment 43. The composition or kit of any one of Embodiments 38 to 42, wherein the target nucleic acid is a pathogen-derived nucleic acid.

Embodiment 44. The composition or kit of Embodiment 43, wherein the pathogen-derived target nucleic acid is a viral nucleic acid.

Embodiment 45. The composition or kit of Embodiment 44, wherein the viral nucleic acid is a human papillomavirus (HPV) nucleic acid.

Embodiment 46. The composition or kit of Embodiment 45, wherein the HPV nucleic acid is selected from the group consisting of HPV types 16 and 59.

Embodiment 47. The composition or kit of Embodiment 45, wherein the composition or kit comprises amplification oligomers for detection of two or more types of HPV.

Embodiment 48. The composition or kit of Embodiment 47, wherein the two or more types of HPV comprise two or more of HPV types 16, 18, 31, 33, 35, 39, 45, 51, 52, 56, 58, 59, 66, and 68.

Embodiment 49. The composition or kit of Embodiment 48, wherein the two or more types of HPV comprise each of HPV type 16 (HPV16) and HPV type 59 (HPV59).

Embodiment 50. The composition or kit of Embodiment 49, wherein the at least two target-specific amplification oligomers comprise (i) an HPV16-specific amplification oligomer comprising a target-hybridizing sequence as shown in SEQ ID NO:2, and (ii) an HPV59-specific amplification oligomer comprising a target-hybridizing sequence as shown in SEQ ID NO:4.

Embodiment 51. The composition or kit of Embodiment 48, wherein the two or more types of HPV comprise each of HPV types 16, 18, 31, 33, 35, 39, 45, 51, 52, 56, 58, 59, 66, and 68.

Embodiment 52. The composition or kit of any one of Embodiments 38 to 51, further comprising (i) at least one control detection probe oligomer configured to specifically hybridize to a nucleolin control amplification product amplifiable by the at least two control amplification oligomers, and (ii) at least one target-specific detection probe oligomer configured to specifically hybridize to a target amplification product amplifiable by the at least two target-specific amplification oligomers.

Embodiment 53. The composition or kit of any one of Embodiments 39 to 41, further comprising (i) at least one control detection probe oligomer configured to specifically hybridize to a control amplification product amplifiable by the at least two control amplification oligomers, and (ii) at least one target-specific detection probe oligomer configured to specifically hybridize to a target amplification product amplifiable by the at least two target-specific amplification oligomers.

Embodiment 54. The composition or kit of Embodiment 53, wherein the at least one control detection probe oligomer comprises a target-hybridizing sequence selected from the group consisting of SEQ ID NO:5, SEQ ID NO:6, and SEQ ID NO:7.

Embodiment 55. The composition or kit of Embodiment 54, wherein the at least one control detection probe oligomer comprises a target hybridizing sequence as shown in SEQ ID NO:6.

Embodiment 56. The composition or kit of Embodiment 52, wherein each of the at least one control detection probe oligomer and the at least one target-specific detection probe oligomer comprises a detectable label.

Embodiment 57. The composition or kit of Embodiment 56, wherein the detectable label is a chemiluminescent or fluorescent label.

Embodiment 58. The composition or kit of Embodiment 57, wherein the detectable label is a chemiluminescent label.

Embodiment 59. The composition or kit of Embodiment 58, wherein the chemiluminescent label is an acridinium ester (AE) compound.

Embodiment 60. The composition or kit of Embodiment 57, wherein the detectable label is a fluorescent label and each of the at least one control detection probe oligomer and the at least one target-specific detection probe oligomer further comprises a non-fluorescent quencher.

Embodiment 61. The composition or kit of Embodiment 53, wherein each of the at least one control detection probe oligomer and the at least one target-specific detection probe oligomer comprises a detectable label.

Embodiment 62. The composition or kit of Embodiment 61, wherein the detectable label is a chemiluminescent or fluorescent label.

Embodiment 63. The composition or kit of Embodiment 62, wherein the detectable label is a chemiluminescent label.

Embodiment 64. The composition or kit of Embodiment 63, wherein the chemiluminescent label is an acridinium ester (AE) compound.

Embodiment 65. The composition or kit of Embodiment 64, wherein the at least one control detection probe oligomer comprises a target-hybridizing sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

Embodiment 66. The composition or kit of Embodiment 65, wherein the at least one control detection probe oligomer comprises a target-hybridizing sequence selected from the group consisting of SEQ ID NO: 10. SEQ ID NO:14, and SEQ ID NO:16.

Embodiment 67. The composition or kit of Embodiment 65, wherein the at least one control detection probe oligomer comprises a target-hybridizing sequence as shown in SEQ ID NO:14.

Embodiment 68. The composition or kit of Embodiment 62, wherein the detectable label is a fluorescent label and each of the at least one control detection probe oligomer and the at least one target-specific detection probe oligomer further comprises a non-fluorescent quencher.

Embodiment 69. A composition or kit for assessing cellularity of a biological sample, the composition or kit comprising: (a) at least two amplification oligomers capable of amplifying a target region of a human nucleolin nucleic acid corresponding to a region of SEQ ID NO:1 from about nucleotide position 585 to about nucleotide position 677, and (b) at least one detection probe oligomer configured to specifically hybridize to a nucleolin amplification product amplifiable by the at least two amplification oligomers.

Embodiment 70. The composition or kit of Embodiment 69, wherein the at least two amplification oligomers comprise (i) a first amplification oligomer comprising a first target-hybridizing sequence that targets a sequence contained with the region of SEQ ID NO:1 from nucleotide position 561 to nucleotide position 627, and/or (ii) a second amplification oligomer comprising a second target-hybridizing sequence that targets a sequence contained with the region of SEQ ID NO:1 from nucleotide position 646 to nucleotide position 677.

Embodiment 71. The composition or kit of Embodiment 70, wherein (i) the first amplification oligomer comprises a first target-hybridizing sequence as shown in SEQ ID NO:2, and/or (ii) the second amplification oligomer comprises a second target-hybridizing sequence as shown in SEQ ID NO:4.

Embodiment 72. The composition or kit of any one of Embodiments 69 to 71, wherein the at least one detection probe oligomer comprises a target-hybridizing sequence selected from the group consisting of SEQ ID NO:5, SEQ ID NO:6, and SEQ ID NO:7.

Embodiment 73. The composition or kit of Embodiment 72, wherein the at least one detection probe oligomer comprises a target-hybridizing sequence as shown in SEQ ID NO:6.

Embodiment 74. The composition or kit of any one of Embodiments 69 to 71, wherein the at least one detection probe oligomer comprises a detectable label.

Embodiment 75. The composition or kit of Embodiment 74, wherein the detectable label is a chemiluminescent or fluorescent label.

Embodiment 76. The composition or kit of Embodiment 75, wherein the detectable label is a chemiluminescent label.

Embodiment 77. The composition or kit of Embodiment 76, wherein the chemiluminescent label is an acridinium ester (AE) compound.

Embodiment 78. The composition or kit of Embodiment 77, wherein the at least one detection probe oligomer comprises a target-hybridizing sequence selected from the group consisting of SEQ ID NO:9, SEQ ID NO:10, SEQ ID NO: 1l, SEQ ID NO:12, SEQ ID NO:13, SEQ ID NO:14, SEQ ID NO:15, SEQ ID NO:16, and SEQ ID NO:17.

Embodiment 79. The composition or kit of Embodiment 78, wherein the at least one detection probe oligomer comprises a target-hybridizing sequence selected from the group consisting of SEQ ID NO:10, SEQ ID NO:14, and SEQ ID NO:16.

Embodiment 80. The composition or kit of Embodiment 78, wherein the at least one detection probe oligomer comprises a target-hybridizing sequence as shown in SEQ ID NO:14.

Embodiment 81. The composition or kit of Embodiment 75, wherein the detectable label is a fluorescent label and the at least one detection probe oligomer further comprises a non-fluorescent quencher.

Embodiment 82. A detection probe oligomer comprising: a nucleolin-specific detection probe target-hybridizing sequence that is from about 15 to about 35 nucleotides in length and is configured to hybridize to a target sequence contained within a target region of human nucleolin nucleic acid corresponding to a region of SEQ ID NO:1 from about nucleotide position 585 to about nucleotide position 677.

Embodiment 83. The detection probe oligomer of Embodiment 82, wherein the detection probe oligomer comprises a target-hybridizing sequence selected from the group consisting of SEQ ID NO:5, SEQ ID NO:6, and SEQ ID NO:7.

Embodiment 84. The detection probe oligomer of Embodiment 83, wherein the detection probe oligomer comprises a target-hybridizing sequence as shown in SEQ ID NO:6.

Embodiment 85. The detection probe oligomer of any one of Embodiments 82 to 84, wherein the detection probe oligomer comprises a detectable label.

Embodiment 86. The detection probe oligomer of Embodiment 85, wherein the detectable label is a chemiluminescent or fluorescent label.

Embodiment 87. The detection probe oligomer of Embodiment 86, wherein the detectable label is a chemiluminescent label.

Embodiment 88. The detection probe oligomer of Embodiment 87, wherein the chemiluminescent label is an acridinium ester (AE) compound.

Embodiment 89. The detection probe oligomer of Embodiment 86, wherein the detectable label is a fluorescent label and the detection probe oligomer further comprises a non-fluorescent quencher.

Embodiment 90. The detection probe oligomer of Embodiment 83, wherein the detection probe oligomer comprises a chemiluminescent label.

Embodiment 91. The detection probe oligomer of Embodiment 90, wherein the chemiluminescent label is an acridinium ester (AE) compound.

Embodiment 92. The detection probe oligomer of Embodiment 91, wherein the detection probe oligomer comprises a target-hybridizing sequence selected from the group consisting of SEQ ID NO:9, SEQ ID NO:10, SEQ ID NO: 11, SEQ ID NO:12, SEQ ID NO:13, SEQ ID NO:14, SEQ ID NO:15, SEQ ID NO:16, and SEQ ID NO:17.

Embodiment 93. The detection probe oligomer of Embodiment 92, wherein the detection probe oligomer comprises a target-hybridizing sequence selected from the group consisting of SEQ ID NO:10, SEQ ID NO:14, and SEQ ID NO:16.

Embodiment 94. The detection probe oligomer of Embodiment 92, wherein the detection probe oligomer comprises a target-hybridizing sequence as shown in SEQ ID NO:14.

Embodiment 95. A method for assessing cellularity of a biological sample for use in a molecular diagnostic assay, the method comprising: (a) providing a biological sample suspected of containing human cells; (b) contacting the sample with at least two amplification oligomers capable of amplifying a target region of a human nucleolin nucleic acid corresponding to a region of SEQ ID NO:1 from about nucleotide position 585 to about nucleotide position 677; (c) performing an in vitro nucleic acid amplification reaction, wherein any nucleolin nucleic acid present in the sample is used as a template for generating an amplification product; and (d) detecting the presence or absence of the amplification product, wherein the detecting step comprises contacting the in vitro nucleic acid amplification reaction with at least one detection probe oligomer configured to specifically hybridize to the amplification product under conditions whereby the presence or absence of the amplification product is determined, and wherein the presence of the amplification product indicates that the cellularity of the sample is adequate for molecular diagnostic testing.

Embodiment 96. The method of Embodiment 95, wherein the at least two amplification oligomers comprise (i) a first amplification oligomer comprising a first target-hybridizing sequence that targets a sequence contained with the region of SEQ ID NO:1 from nucleotide position 561 to nucleotide position 627, and/or (ii) a second amplification oligomer comprising a second target-hybridizing sequence that targets a sequence contained with the region of SEQ ID NO:1 from nucleotide position 646 to nucleotide position 677.

Embodiment 97. The method of Embodiment 96, wherein (i) the first amplification oligomer comprises a first target-hybridizing sequence as shown in SEQ ID NO:2, and/or (ii) the second amplification oligomer comprises a second target-hybridizing sequence as shown in SEQ ID NO:4.

Embodiment 98. The method of any one of Embodiments 95 to 97, wherein the sample is a tissue swab.

Embodiment 99. The method of Embodiment 98, the tissue swab is a cervical swab, a vaginal swab, a nasal swab, a buccal swab, a throat swab, an anal swab, a urethral/penile swab, or a swab from an open wound/sore/laceration.

Embodiment 100. The method of any one of Embodiments 95 to 99, wherein the at least one detection probe oligomer comprises a target-hybridizing sequence selected from the group consisting of SEQ ID NO:5, SEQ ID NO:6, and SEQ ID NO:7.

Embodiment 101. The method of Embodiment 100, wherein the at least one detection probe oligomer comprises a target-hybridizing sequence as shown in SEQ ID NO:6.

Embodiment 102. The method of any one of Embodiments 95 to 99, wherein the at least one detection probe oligomer comprises a detectable label.

Embodiment 103. The method of Embodiment 102, wherein the detectable label is a chemiluminescent or fluorescent label.

Embodiment 104. The method of Embodiment 103, wherein the detectable label is a chemiluminescent label.

Embodiment 105. The method of Embodiment 1104, wherein the chemiluminescent label is an acridinium ester (AE) compound.

Embodiment 106. The method of Embodiment 105, wherein the at least one detection probe oligomer comprises a target-hybridizing sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

Embodiment 107. The method of Embodiment 106, wherein the at least one detection probe oligomer comprises a target-hybridizing sequence selected from the group consisting of SEQ ID NO:10, SEQ ID NO:14, and SEQ ID NO:16.

Embodiment 108. The method of Embodiment 106, wherein the at least one detection probe oligomer comprises a target-hybridizing sequence as shown in SEQ ID NO:14.

Embodiment 109. The method of Embodiment 103, wherein the detectable label is a fluorescent label and the at least one detection probe oligomer further comprises a non-fluorescent quencher.

Embodiment 110. The method of any one of Embodiments 95 to 103, wherein the in vitro nucleic acid amplification reaction is an isothermal amplification reaction.

Embodiment 111. The method of Embodiment 110, wherein the isothermal amplification reaction is a transcription-mediated amplification reaction.

Embodiment 112. The method of any one of Embodiments 95 to 111, wherein if the presence of the amplification product is determined, then the sample is used in a molecular diagnostic assay to determine the presence or absence of a target nucleic acid in the sample.

Embodiment 113. The method of Embodiment 112, wherein the target nucleic acid is an RNA.

Embodiment 114. The method of Embodiment 112 or 113, wherein the target nucleic acid is a pathogen-derived nucleic acid.

Embodiment 115. The method of Embodiment 114, wherein the pathogen-derived nucleic acid is a viral nucleic acid.

Embodiment 116. The method of Embodiment 115, wherein the viral nucleic acid is a human papillomavirus (HPV) nucleic acid.

These and other aspects of the invention will become evident upon reference to the following detailed description of the invention.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

“Sample” includes any specimen that may contain a target nucleic acid. Samples include “biological samples,” which include any tissue or material derived from a living or dead human. The biological sample may be treated to physically or mechanically disrupt tissue or cell structure, thus releasing intracellular components into a solution which may further contain enzymes, buffers, salts, detergents, and the like, which are used to prepare a biological sample for analysis. Also, samples may include processed samples such as samples in which one or more components have been concentrated or purified. Processed samples include, e.g., those obtained from passing samples over or through a filtering device, or following centrifugation, or by adherence to a medium, matrix, or support.

The term “cellularity” as used herein is a quantitative term referring to the relative amount of eukaryotic (e.g., human) cells or cellular-derived material in a sample. Sufficient or adequate cellularity of a sample means that the number of cells or amount of cellular-derived material in a sample is sufficient for performing nucleic acid-based molecular testing on the sample.

A “nucleotide” as used herein is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose.

“Nucleic acid” and “polynucleotide” refer to a multimeric compound comprising nucleotides and/or nucleotide analogs linked together to form a biopolymer. The biopolymers include conventional RNA, conventional DNA, mixed RNA-DNA, and nucleotide analog containing versions thereof. A nucleic acid “backbone” may be made up of a variety of linkages, including one or more of sugar-phosphodiester linkages, peptide-nucleic acid bonds (“peptide nucleic acids” or PNA), phosphorothioate linkages, methylphosphonate linkages, or combinations thereof. Sugar moieties of a nucleic acid may be ribose, deoxyribose, or similar compounds with substitutions. e.g., analogs with a methoxy, fluoro or halide group at the 2′ position of the ribose (also referred to herein as “2′-O-Me” or “2′-methoxy” or 2′-fluoro, or “2′-halide”). Nitrogenous bases may be conventional bases, adenine (A), uracil (U), guanine (G), thymine (T), and cytosine (C), and analogs thereof (e.g., inosine, 5 methyl 2′ deoyxcytosine (“5-Me-dC” or “5MeC”), and isoguanine). Nucleic acids may include one or more “abasic” residues where the backbone includes no nitrogenous base for position(s) of the polymer.

“Oligomer.” “oligonucleotide,” or “oligo” refers to a nucleic acid of generally less than 1,000 nucleotides (nt), including those in a size range having a lower limit of about 5 nt and an upper limit of about 900 nt. The term oligonucleotide does not denote any particular function to the reagent; rather, it is used generically to cover all such reagents described herein. Oligomers may be referred to by a functional name (e.g., capture probe, detection probe, primer, or promoter primer) but those skilled in the art will understand that such terms refer to oligomers.

A “target nucleic acid” as used herein is a nucleic acid comprising a target sequence to be amplified. Target nucleic acids may be DNA or RNA and may be either single-stranded or double-stranded. The target nucleic acid may include other sequences besides the target sequence, which may not be amplified.

The term “target sequence” or “target nucleic acid sequence” as used herein refers to the particular nucleotide sequence of the target nucleic acid that is to be amplified and/or detected. The “target sequence” includes the complexing sequences to which oligonucleotides (e.g., priming oligonucleotides and/or promoter oligonucleotides) complex during an amplification processes (e.g., PCR, TMA). Unless the context clearly dictates otherwise, where the target nucleic acid is originally single-stranded, the term “target sequence” will also refer to the sequence complementary to the “target sequence” as present in the target nucleic acid, and where the target nucleic acid is originally double-stranded, the term “target sequence” refers to both the sense (+) and antisense (−) strands.

“Target-hybridizing sequence” is used herein to refer to the portion of an oligomer that is configured to hybridize with a target nucleic acid sequence. Preferably, the target-hybridizing sequences are configured to specifically hybridize with a target nucleic acid sequence. Target-hybridizing sequences may be 100% complementary to the portion of the target sequence to which they are configured to hybridize; but not necessarily. Target-hybridizing sequences may also include inserted, deleted and/or substituted nucleotide residues relative to a target sequence. Less than 100% complementarity of a target-hybridizing sequence to a target sequence may arise, for example, when the target nucleic acid is a plurality of strains within a species. It is understood that other reasons exist for configuring a target-hybridizing sequence to have less than 100% complementarity to a target nucleic acid.

Oligomer target-hybridizing sequences defined herein by reference to a specific sequence (e.g., by reference a region within SEQ ID NO:1) are also understood to include functional complements thereof, unless the context clearly dictates otherwise. Thus, for example, where a target-hybridizing sequence for a detection probe oligomer is defined by reference to a specific sequence, it is understood that the detection probe may include a corresponding detection probe oligomer having a target-hybridizing sequence that is the complement of the specific reference sequence; or where a detection probe oligomer is defined by its configuration to hybridize to a specific sequence, it is understood that the detection probe may include a corresponding detection probe oligomer having a target-hybridizing sequence that is configured to hybridize to the complement of the specific reference sequence.

The term “region,” as used herein, refers to a portion of a nucleic acid wherein said portion is smaller than the entire nucleic acid. For example, when the nucleic acid is a nucleolin gene sequence or a target nucleic acid for detection, the term “region” may be used to refer to a smaller area of the nucleic acid, wherein the smaller area is targeted by one or more oligonucleotides of the invention (e.g., a “target region” to be amplified). As another non-limiting example, when the nucleic acid in reference is an oligonucleotide promoter primer, the term “region” may be used to refer to the smaller promoter portion of the entire oligonucleotide. As yet another non-limiting example, when the nucleic acid in reference is an amplicon, the term region may be used to refer to the smaller nucleotide sequence identified for hybridization by the target-hybridizing sequence of a probe.

A “nucleic-acid-based assay,” as used herein, is an assay for the detection of a target sequence within a target nucleic acid and utilizing one or more oligonucleotides that specifically hybridize to the target sequence. In certain embodiments in accordance with the present invention, a nucleic-acid-based assay is an “amplification assay,” i.e., an assay that utilizes one or more steps for amplifying a nucleic acid target sequence.

“Nucleic acid amplification” refers to any well-known in vitro procedure that produces multiple copies of a target nucleic acid sequence. Examples of such procedures include transcription-associated methods, e.g., transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), replicase-mediated amplification (e.g., U.S. Pat. No. 4,786,600), polymerase chain reaction (PCR) (e.g., U.S. Pat. Nos. 4,683,195, 4,683,202, and 4,800,159), ligase chain reaction (LCR) (e.g., EP Patent No. 0320308), and strand-displacement amplification (SDA) (e.g., U.S. Pat. No. 5,422,252).

By “amplicon” or “amplification product” is meant a nucleic acid molecule generated in a nucleic acid amplification reaction and which is derived from a target nucleic acid. An amplicon or amplification product contains a target nucleic acid sequence that may be of the same or opposite sense as the target nucleic acid.

An “amplification oligonucleotide” or “amplification oligomer” is an oligonucleotide that hybridizes to a target nucleic acid and participates in a nucleic acid amplification reaction, e.g., serving as a primer. Amplification oligomers can have 3′ ends that are extended by polymerization as part of the nucleic acid amplification reaction. Amplification oligomers can alternatively have 3′ ends that are not extended by polymerization, but provide a component that facilitates nucleic acid amplification, e.g., a promoter sequence joined 5′ to the target-specific sequence of the amplification oligomer. Such an amplification oligomer is referred to as a promoter provider. Amplification oligomers that provide both a 3′ target-specific sequence that is extendable by polymerization and a 5′ promoter sequence are referred to as promoter primers. Amplification oligomers may be optionally modified to include 5′ non-target-specific sequences such as tags, promoters (as mentioned), or other sequences used or useful for manipulating or amplifying the primer or target oligonucleotide.

“Detection probe oligomer.” “detection probe,” or “probe” refers to an oligomer that hybridizes specifically to a target sequence, including an amplified product, under conditions that promote nucleic acid hybridization, for detection of the target nucleic acid. Detection may either be direct (i.e., probe hybridized directly to the target) or indirect (i.e., a probe hybridized to an intermediate structure that links the probe to the target). A probe's target-specific sequence generally refers to the specific sequence within a larger sequence to which the probe hybridizes specifically. A detection probe may include target-specific sequence(s) and non-target-specific sequence(s). Such non-target-specific sequences can include sequences which will confer a desired secondary or tertiary structure, such as a hairpin structure, which can be used to facilitate detection and/or amplification.

“Label” or “detectable label” refers to a moiety or compound joined directly or indirectly to a probe that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s), which amplify a detectable signal). Any detectable moiety may be used, e.g., radionuclide, ligand such as biotin or avidin, enzyme, enzyme substrate, reactive group, chromophore such as a dye or particle (e.g., latex or metal bead) that imparts a detectable color, luminescent compound (e.g., bioluminescent, phosphorescent, or chemiluminescent compound such as an acridiniumn ester (“AE”) compound), and fluorescent compound (i.e., fluorophore). Fluorophores may be used in combination with a quencher molecule that absorbs light when in close proximity to the fluorophore to diminish background fluorescence. Detectably labeled probes include, for example, hydrolysis (e.g., TaqMan™) probes, AE-labeled probes, molecular torches, and molecular beacons.

The term “configured to” denotes an actual arrangement of the polynucleotide sequence configuration of a referenced oligonucleotide target-hybridizing sequence. For example, amplification oligomers that are configured to generate a specified amplicon from a target sequence have polynucleotide sequences that hybridize to the target sequence and can be used in an amplification reaction to generate the amplicon. Also, as an example, oligonucleotides that are configured to specifically hybridize to a target sequence have a polynucleotide sequence that specifically hybridizes to the referenced sequence under stringent hybridization conditions.

“Pathogens” include viruses, bacteria, protozoa, fungi, and other microorganisms responsible for disease in humans and other animals.

Reference to a numerical range herein (e.g., “X to Y” or “from X to Y”) includes the endpoints defining the range and all values falling within the range.

Unless otherwise apparent from the context, when a value is expressed as “about” X or “approximately” X, the stated value of X will be understood to be accurate to ±10%. When the phrase “about nucleotide position” X is used in reference to the nucleolin gene sequence of SEQ ID NO:1, the stated nucleotide position of X will be understood to mean a nucleotide position within 25 nucleotides upstream or downstream of X.

### DESCRIPTION

The present invention provides compositions and methods for performing a nucleic-acid-based assay for determining the presence or absence of a target nucleic acid in a biological sample. The nucleic-acid-based assays in accordance with the present disclosure are generally directed to the detection of target nucleic acids that may be contained within a sample containing eukaryotic (e.g., human) cells or cellular-derived material. The target nucleic acid may, for example, be a nucleic acid derived from a pathogen such as, for example, a viral or bacterial pathogen. The compositions and methods are based, in part, on the use of a nucleolin gene sequence as a control for assessing the cellularity of a sample and thereby determining whether the sample to be tested contains sufficient eukaryotic (e.g., human) cells or cellular-derived material for performing the nucleic-acid-based assay. In related aspects, the present invention provides compositions and methods for assessing cellularity of a biological sample for use in a molecular diagnostic assay by detecting the presence or absence of a nucleolin nucleic acid in the sample.

A method for performing a nucleic-acid-based assay to determine the presence or absence of a target nucleic acid in a biological sample generally includes the following steps: (a) providing a biological sample suspected of containing eukaryotic cells that may comprise the target nucleic acid; (b) contacting the sample with at least one control oligomer and at least one target-specific oligomer, wherein the at least one control oligomer is capable of specifically hybridizing to a target sequence of a nucleolin control nucleic acid, and wherein the at least one target-specific oligomer is capable of specifically hybridizing to a target sequence of the target nucleic acid; and (c) performing an in vitro nucleic-acid-based assay utilizing the at least one control oligomer and at least one target-specific oligomer to detect the presence or absence of each of the nucleolin control nucleic acid and the target nucleic acid, respectively. If the nucleolin control nucleic acid is present, then the presence of the eukaryotic cells in the sample is assured, and any negative result for detection of the target nucleic acid is distinguished from a negative result due to the absence of eukaryotic cells or cellular-derived material in the sample. The nucleic-acid-based assay utilizing the at least one control oligomer may be performed as an exogenous assay system (i.e., as a stand-alone assay for detecting the nucleolin control nucleic acid that is run in parallel with the nucleic-acid-based assay for detecting the target nucleic acid) or as an endogenous system (i.e., wherein the nucleic-acid-based assay utilizing the at least one control oligomer is incorporated with the nucleic-acid-based assay for detecting the target nucleic acid in a multiplex format.

In some embodiments of the above method, the nucleic-acid-based assay is an amplification assay, wherein the presence or absence of each of the nucleolin control nucleic acid and the target nucleic acid is determined by detecting the presence or absence of a corresponding amplification product generated by a nucleic acid amplification reaction. Embodiments comprising an amplification assay generally include the following steps: (a) providing a biological sample suspected of containing cukaryotic cells that may comprise the target nucleic acid; (b) contacting the sample with at least two control amplification oligomers and at least two target-specific amplification oligomers, wherein the at least two control amplification oligomers are capable of amplifying a target region of a nucleolin control nucleic acid, and wherein the at least two target-specific amplification oligomers are capable of amplifying a target region of the target nucleic acid; (c) performing an in vitro nucleic acid amplification reaction, wherein any nucleolin control nucleic acid present in the sample is used as a template for generating a control amplification product, and wherein any target nucleic acid present in the sample is used as a template for generating a target amplification product; and (d) detecting the presence or absence of each of the control amplification product and the target amplification product. If the control amplification product is present, then the presence of the eukaryotic cells in the sample is assured and the presence or absence of the target amplification product determines, respectively, the presence or absence of the target nucleic acid in the sample. A variety of known nucleic acid amplification reactions may be used, including, for example, transcription-associated amplification (e.g., transcription-mediated amplification (TMA) or nucleic acid sequence-based amplification (NASBA)), Polymerase Chain Reaction (PCR), replicase-mediated amplification, and Ligase Chain Reaction (LCR), to name a few. In some preferred embodiments, the nucleic acid amplification reaction is an isothermal amplification reaction such as, for example, transcription-mediated amplification.

The eukaryotic cells may be, for example, from any species of animal. In typical variations, the eukaryotic cells are typically mammalian cells such as, e.g., human cells. Particularly suitable samples for use in accordance with the present disclosure include tissue swabs. In certain variations, the tissue swab is selected from a cervical swab, a vaginal swab, a nasal swab, a buccal swab, a throat swab, an anal swab, a urethral/penile swab, and a swab from an open wound/sore/laceration.

In some embodiments, the at least one control oligomer targets a sequence of a human nucleolin control nucleic acid corresponding to SEQ ID NO:1. For example, in certain variations, the at least one control oligomer targets a sequence within a region of human nucleolin nucleic acid corresponding to a region of SEQ ID NO:1 from about nucleotide position 585 to about nucleotide position 677 (e.g., a region of SEQ ID NO:1 from nucleotide position X to nucleotide position 677, wherein X is a nucleotide position selected from 561, 564, 579, 582, 585, 588, 603, and 606). In some variations comprising an amplification assay, the at least two control amplification oligomers for amplifying a target region of the nucleolin nucleic acid are capable of amplifying a nucleolin nucleic acid target region as defined above; in some such embodiments, the at least two control amplification oligomers comprise (i) a first control amplification oligomer comprising a first target-hybridizing sequence that targets a sequence contained with the region of SEQ ID NO:1 from nucleotide position 561 to nucleotide position 627, and/or (ii) a second control amplification oligomer comprising a second target-hybridizing sequence that targets a sequence contained with the region of SEQ ID NO:1 from nucleotide position 646 to nucleotide position 677. In more particular variations, the first control amplification oligomer comprises a first target-hybridizing sequence as shown in SEQ ID NO:2, and/or the second control amplification oligomer comprises a second target-hybridizing sequence as shown in SEQ ID NO:4.

In certain embodiments, the target nucleic acid for detection is an RNA. Various types of RNA nucleic acids can be detected. The RNA can be ribosomal RNA (rRNA), messenger RNA (mRNA), or heterogeneous nuclear RNA (hnRNA). In some variations, a pathogen-derived nucleic acid is ribosomal RNA (e.g., 18S rRNA, 5S rRNA, 5.8S rRNA, or 28S rRNA).

Preferably, the target nucleic acid for detection is from a pathogen such as, for example, a viral pathogen. Exemplary viral pathogens include, e.g., human papillomavirus (HPV) (for example, any one or more of HPV types 16, 18, 31, 33, 35, 39, 45, 51, 52, 56, 58, 59, 66, and 68 or types within one or more of HPV groups A1, A2, C1, and D). In other embodiments, the viral pathogen is H1N1, H2N2. H3N2, H1Nlpdm09, severe acute respiratory syndrome coronavirus 1 (SARS-CoV-1), severe acute respiratory syndrome coronavirus 2 (SARS-CoV-2), Middle East respiratory syndrome (MERS), or influenza (e.g., any of Influenza A, Influenza B, Influenza C, or Influenza D). In some embodiments, a viral pathogen for detection is a DNA virus such as, e.g., a virus from the family Adenoviridae, Papovavirndae, Parvoviridae, Herpesviridae, Poxviridae, Anelloviridae, or Pleolipoviridae. In other embodiments, the viral pathogen is an RNA virus such as, e.g., a virus from a family selected from Reoviridae. Picornaviridae. Caliciviridae. Togaviridae, Arenaviridae, Flaviviridae, Orthomyxoviridae, Paramyxoviridae. Bunyaviridae, Rhabdoviridae, Filoviridae, Coronaviridae, Astroviridae. and Bornaviridae. In yet other embodiments, the pathogen is a reverse transcribing virus such as, e.g., a virus from a family selected from Retroviridae, Caulimoviridae, and Hepadnaviridae. More generally, a viral target can be a virus categorized by the Baltimore classification; in certain embodiments, the viral target is an RNA virus (e.g., Influenza A, Zika, Hepatitis C), a DNA virus (e.g., HPV, Epstein Barr, Smallpox), a positive sense RNA virus (e.g., Hepatitis A, rubella), a negative sense RNA virus (e.g., Ebola, measles, mumps), a dsDNA virus (e.g., HPV, chickenpox, herpes), an ssDNA virus, a dsRNA virus (e.g., a rotavirus), a positive-strand ssRNA virus, a negative-strand ssRNA virus, an ssRNA-RT virus (e.g., retroviruses), or a ds-DNA-RT virus (e.g., Hepatitis B).

In some embodiments wherein the pathogen target is a bacterial target, the bacterial target is from the genus Acinetobacter, Arcanobacterium, Bacillus, Bartonella, Bordetella, Borrelia, Brucella, Campylobacter, Chlamydia, Chlamydophila, Citrobacter, Clostridium, Corynebacterium, Enterococcus, Escherichia, Francisella, Globicatella, Haemophilus, Helicobacter, Klebsiella, Kluyvera, Legionella, Leptospira, Listeria, Morganella, Mycobacterium, Mycoplasma, Myroides, Neisseria, Proteus, Pseudomonas, Ralstonia, Rickettsia, Salmonella, Serratia, Shigella, Staphylococcus, Streptococcus, Treponema, Ureaplasma, Vibrio, or Yersinia.

In other variations comprising detection of a pathogen-derived target, the pathogen target is a protist target. Exemplary protist targets include protists from the genus Babesia, Plasmodium. Trypanosoma, Leishmania, Anaplasma, or Toxoplasma.

In certain embodiments wherein the target nucleic acid is an HPV nucleic acid, the at least one target-specific oligomer (e.g., at least two HPV-specific amplification oligomers) target the E6/E7 gene of HPV. In some variations, the HPV target nucleic acid is selected from HPV types 16 and 59. In some embodiments, the nucleic-acid-based assay for detection of HPV is a multiplex assay (e.g., multiplex amplification assay) comprising detection of two or more types of HPV. For example, the two or more types of HPV for detection in a multiplex assay may comprises two or more of (e.g., each of) HPV types 16, 18, 31, 33, 35, 39, 45, 51, 52, 56, 58, 59, 66, and 68. In some multiplex embodiments, the two or more types of HPV comprise each of HPV type 16 (HPV16) and HPV type 59 (HPV59); in some such variations, the nucleic-acid-based assay is an amplification assay wherein the at least two target-specific amplification oligomers include an HPV16-specific amplification oligomer comprising a target-hybridizing sequence as shown in SEQ ID NO:2 and an HPV59-specific amplification oligomer comprising a target-specific sequence as shown in SEQ ID NO:4.

In some preferred embodiments of a method for performing an amplification assay for detection of an HPV target nucleic acid as above, the detection of the nucleolin control nucleic acid is performed endogenously with the detection of the HPV target nucleic acid (i.e., the in vitro nucleic acid amplification reaction generating the control amplification product and the detection of the control amplification product are performed in a multiplex format with the generation and detection of the target amplification product). In some such embodiments, the amplification assay includes amplification and detection of HPV16 and HPV59 target sequences (e.g., amplification and detection of HPV16 and HPV59 E6/E7 target sequences). Particularly suitable amplification oligomers for performing nucleolin detection as an endogenous system with detection of HPV include (i) a first amplification oligomer comprising a target-hybridizing sequence as shown in SEQ ID NO:2, (ii) a second amplification oligomer comprising a target-hybridizing sequence as shown in SEQ ID NO:4, (iii) a third amplification oligomer comprising a target-hybridizing sequence that hybridizes specifically to an HPV16 E6/E7 target sequence, wherein the first and third amplification oligomers are capable of amplifying an HPV16 E6/E7 target region, and (iv) a fourth amplification oligomer comprising a target-hybridizing sequence that hybridizes specifically to an HPV59 E6/E7 target sequence, wherein the second and fourth amplification oligomers are capable of amplifying an HPV59 E6/E7 target region. In some embodiments wherein the amplification and detection of the control amplification product and HPV target amplification product are performed in multiplex format, the amplification assay is for the detection of two or more of HPV types 16, 18, 31, 33, 35, 39, 45, 51, 52, 56, 58, 59, 66, and 68 (e.g., amplification and detection of at least HPV16 and HPV59 and optionally one or more of HPV types 18, 31, 33, 35, 39, 45, 51, 52, 56, 58, 66, and 68; or amplification and detection of each of HPV types 16, 18, 31, 33, 35, 39, 45, 51, 52, 56, 58, 59, 66, and 68).

In certain embodiments, a method for performing an amplification assay as described herein further includes purifying the control and target nucleic acids from other components in the sample before amplification. Such purification may include methods of separating and/or concentrating organisms contained in a sample from other sample components, or removing or degrading non-nucleic acid sample components, e.g., protein, carbohydrate, salt, lipid, etc. In particular embodiments, control and target nucleic acids are captured specifically or non-specifically and separated from other sample components. Target capture typically occurs in a solution phase mixture that contains one or more capture probe oligomers that hybridize to a target sequence of the control or target nucleic acid under hybridizing conditions. For embodiments comprising a capture probe tail, a target:capture-probe complex is captured by using hybridization conditions under which the capture probe tail hybridizes to an immobilized probe. Certain embodiments use a particulate solid support, such as paramagnetic beads. Selective and non-specific target capture methods are also described, e.g., in U.S. Pat. No. 6,110,678 and International Patent Application Pub. No. WO 2008/016988, each incorporated by reference herein.

A detection step may be performed using any of a variety of known techniques to detect a signal specifically associated with the amplified control or target sequence, such as, e.g., by hybridizing the amplification product with a labeled detection probe and detecting a signal resulting from the labeled probe (including from label released from the probe following hybridization in some embodiments). In some embodiments, the labeled probe comprises a second moiety, such as a quencher or other moiety that interacts with the first label, as discussed above. The detection step may also provide additional information on the amplified sequence, such as, e.g., all or a portion of its nucleic acid base sequence. Detection may be performed after the amplification reaction is completed or may be performed simultaneously with amplifying the target region, e.g., in real time. In embodiments that detect the amplified product near or at the end of the amplification step, a linear detection probe may be used to provide a signal to indicate hybridization of the probe to the amplified product. One example of such detection uses a luminescently labeled probe that hybridizes to target nucleic acid (e.g., a chemiluminescent label such as, for example, an acridinium ester (AE) compound). The luminescent label is then hydrolyzed from non-hybridized probe. Detection is performed by chemiluminescence using a luminometer. (See, e.g., International Patent Application Pub. No. WO 89/002476, incorporated by reference herein). In other embodiments that use real-time detection, the detection probe may be a hairpin probe such as, for example, a molecular beacon, molecular torch, or hybridization switch probe that is labeled with a reporter moiety that is detected when the probe binds to amplified product (e.g., a dual-labeled hairpin probe comprising both a fluorescent label and a quenching moiety). In other embodiments for real-time detection, the detection probe is a linear oligomer such as, e.g., an oligomer labeled with both a fluorophore and a quenching moiety (e.g., a TaqMan probe). Such probes may comprise target-hybridizing sequences and non-target-hybridizing sequences. Various forms of such probes have been described previously (see, e.g., U.S. Pat. Nos. 5,210,015; 5,487,972; 5,118.801; 5,312,728; 5,925,517; 6,150,097; 6,849,412; 6,835,542; 6,534,274; and 6,361,945; as well as US Patent Application Pub. Nos. 20060068417A1 and 20060194240A1; each incorporated by reference herein).

Accordingly, in certain variations comprising an amplification assay, the detecting step comprises contacting the in vitro nucleic acid amplification reaction with (i) at least one control detection probe oligomer configured to specifically hybridize to the control amplification product under conditions whereby the presence or absence of the control amplification product is determined, and (ii) at least one target-specific detection probe oligomer configured to specifically hybridize to the target amplification product under conditions whereby the presence or absence of the target amplification product is determined, thereby detecting the presence or absence of each of the control amplification product and the target amplification product. In some embodiments comprising amplification of a human nucleolin target region corresponding to a region of SEQ ID NO:1 from about nucleotide position 585 to about nucleotide position 677 (e.g., utilizing first and second control amplification oligomers comprising target-hybridizing sequences as shown in SEQ ID NO:2 and SEQ ID NO:4), the at least one control detection probe oligomer comprising a target-hybridizing sequence selected from SEQ ID NO:5. SEQ ID NO:6, and SEQ ID NO:7. In some embodiments, each of the at least one control detection probe oligomer and that least one target-specific detection probe oligomer includes a detectable label (e.g., a chemiluminescent label such as, for example, an acridinium ester (AE) compound, or a fluorescent label). In some embodiments wherein the control detection probe oligomer comprises a target-hybridizing sequence selected from SEQ ID NO:5, SEQ ID NO:6, and SEQ ID NO:7, and wherein the control detection probe oligomer comprises an AE label, the at least one control detection probe oligomer comprises a target-hybridizing sequence selected from SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

In related aspects, the present invention provides methods for assessing the cellularity of a biological sample for use in a molecular diagnostic assay. Such methods generally include the following steps: (a) providing a biological sample suspected of containing human cells; (b) contacting the sample with at least two amplification oligomers capable of amplifying a target region of a human nucleolin nucleic acid corresponding to a region of SEQ ID NO:1 from about nucleotide position 585 to about nucleotide position 677; (c) performing an in vitro nucleic acid amplification reaction, wherein any nucleolin nucleic acid present in the sample is used as a template for generating an amplification product; and (d) detecting the presence or absence of the amplification product, wherein the detecting step comprises contacting the in vitro nucleic acid amplification reaction with at least one detection probe oligomer configured to specifically hybridize to the amplification product under conditions whereby the presence or absence of the amplification product is determined, and wherein the presence of the amplification product indicates that the cellularity of the sample is adequate for molecular diagnostic testing. In various embodiments, a method for assessing the cellularity of a biological sample as above may be performed in accordance with embodiments as described above for amplifying and detecting a target region of a human nucleolin target region corresponding to a region of SEQ ID NO:1 from about nucleotide position 585 to about nucleotide position 677. The method may be performed either in the presence or absence of steps for performing a molecular diagnostic test (e.g., a nucleic-acid-based assay to detect a target nucleic acid). In some embodiments further comprising steps for performing the molecular diagnostic test, the molecular diagnostic test may be performed either in a multiplex format or as a separate assay (e.g., in parallel with the cellularity assessment assay or following a determination that the cellularity of the sample is adequate for molecular diagnostic testing).

In other related aspects, the present invention provides a composition or kit for performing an amplification assay to determine the presence or absence of a target nucleic acid in a sample. The composition or kit for performing an amplification assay generally includes at least two control amplification oligomers and at least two target-specific amplification oligomers, wherein the at least two control amplification oligomers are capable of amplifying a target region of a nucleolin control nucleic acid, and wherein the at least two target-specific amplification oligomers are capable of amplifying a target region of the target nucleic acid. In various embodiments, the composition or kit includes an oligomer combination as described above for performing an amplification assay to determine the presence or absence of target nucleic acid in a biological sample.

In other related aspects, the present invention provides a composition or kit for assessing cellularity of a biological sample. The composition or kit generally includes at least two amplification oligomers capable of amplifying a target region of a human nucleolin nucleic acid corresponding to a region of SEQ ID NO:1 from about nucleotide position 585 to about nucleotide position 677, and at least one detection probe oligomer configured to specifically hybridize to a nucleolin amplification product amplifiable by the at least two amplification oligomers. In various embodiments, the composition or kit includes an oligomer combination as described above for assessing cellularity of a biological sample.

In yet other related aspects, the present invention provides a detection probe oligomer as described herein. In certain embodiments, the detection probe oligomer comprises a nucleolin-specific detection probe target-hybridizing sequence that is from about 15 to about 35 nucleotides in length and is configured to hybridize to a target sequence contained within a target region of human nucleolin nucleic acid corresponding to a region of SEQ ID NO:1 from about nucleotide position 585 to about nucleotide position 677. In some variations, the detection probe target-hybridizing sequence is configured to hybridize to a target sequence contained within a region of SEQ ID NO:1 from nucleotide position X to nucleotide position 677, wherein X is a nucleotide position selected from 561, 564, 579, 582, 585, 588, 603, and 606. In more particular embodiments, the detection probe oligomer comprises a target-hybridizing sequence selected from SEQ ID NO:5, SEQ ID NO:6, and SEQ ID NO:7. The detection probe oligomer may include a detectable label (e.g., a chemiluminescent label such as, for example, an acridinium ester (AE) compound, or a fluorescent label). In some embodiments wherein the detection probe oligomer comprises a target-hybridizing sequence selected from SEQ ID NO:5, SEQ ID NO:6, and SEQ ID NO:7, and wherein the detection probe oligomer comprises an AE label, the detection probe oligomer comprises a target-hybridizing sequence selected from SEQ ID NO:9. SEQ ID NO:10, SEQ ID NO:11, SEQ ID NO:12, SEQ ID NO:13, SEQ ID NO:14, SEQ ID NO:15, SEQ ID NO:16, and SEQ ID NO:17. In certain aspects, the present invention provides a kit comprising a detection probe oligomer as described herein.

In certain variations of a composition comprising one or more oligomers as described herein, the composition is a reaction mixture for performing a nucleic-acid-based assay (e.g., an amplification assay). A reaction mixture may further include one or more optional components such as, for example, capture probes, e.g., poly-(k) capture probes as described in US 2013/0209992, which is incorporated herein by reference. For an amplification reaction mixture, the reaction mixture will typically include other reagents suitable for performing in vitro amplification such as, e.g., buffers, salt solutions, appropriate nucleotide triphosphates (e.g., dATP, dCTP, dGTP, and dTTP; and/or ATP, CTP, GTP and UTP), and/or enzymes (e.g., a thermostable DNA polymerase, or reverse transcriptase and/or RNA polymerase), and will typically include test sample components, in which a target nucleic acid may or may not be present. A reaction mixture may include amplification oligomers for only one target nucleic acid target region, or it may include amplification oligomers for multiple target nucleic acid target regions (e.g., amplification oligomers for multiple HPV target regions). In addition, for a reaction mixture that includes a detection probe together with an amplification oligomer combination, selection of amplification oligomers and detection probe oligomers for a reaction mixture are linked by a common target region (i.e., the reaction mixture will include a probe that binds to a sequence amplifiable by an amplification oligomer combination of the reaction mixture). In some embodiments, a reaction mixture comprises an aqueous formulation as described above.

A kit comprising one or more oligomers as described herein may further include one or more optional components for performing a nucleic-acid-based assay. For example, a kit in accordance with the present disclosure may include one or more capture probes, e.g., poly-(k) capture probes as described in US 2013/0209992. A kit for performing an amplification assay may include other reagents suitable for performing in vitro amplification such as, e.g., buffers, salt solutions, appropriate nucleotide triphosphates (e.g., dATP, dCTP, dGTP, dTTP; and/or ATP, CTP, GTP and UTP), and/or enzymes (e.g., a thermostable DNA polymerase, or a reverse transcriptase and/or RNA polymerase). Oligomers as described herein may be packaged in a variety of different embodiments, and those skilled in the art will appreciate that the disclosure embraces many different kit configurations. For example, a kit may include amplification oligomers for only one target nucleic acid target region, or it may include amplification oligomers for multiple target nucleic acid target regions (e.g., amplification oligomers for multiple HPV target regions). In addition, for a kit that includes a detection probe together with an amplification oligomer combination, selection of amplification oligomers and detection probe oligomers for a kit are linked by a common target region (i.e., the kit will include a probe that binds to a sequence amplifiable by an amplification oligomer combination of the kit). In certain embodiments, the kit further includes a set of instructions for practicing methods in accordance with the present disclosure, where the instructions may be associated with a package insert and/or the packaging of the kit or the components thereof.

The compositions, kits, formulations, reaction mixtures, and methods are further illustrated by the following non-limiting examples.

### EXAMPLES

The following examples are provided to illustrate certain disclosed embodiments and are not to be construed as limiting the scope of this disclosure in any way.

### Example 1

Evidence generated during the development of the Aptima HPV assay showed that human RNAs of unknown identity from human cells in Thinprep samples are captured and amplified but not detected by the assay. Studies performed at the time showed that two HPV amplification primers (HPV16 nonT7 and HPV59 T7) were responsible for this non-specific amplification.

To identify the cell RNAs amplified by the Aptima HPV assay, Illumina NGS was performed on Aptima HPV TMA reaction products from three Thinprep samples chosen at random from the Hologic Biobank, using Illumina Mi-Seq sequencing adapters containing the HPV 16 and 59 target binding sequences. This allowed for the amplification and sequencing of only those RNA amplicons that were generated as specific side-reaction products in the assay amp system.

The sequencing results showed several human RNA sequences are amplified by the type 16/59 primer pair, including two mRNAs in human cells, nucleolin and tubulin beta-4B.

### Example 2

Methoxy RNA probes targeting each of the nucleolin and tubulin beta-4B amplicons from Example 1 were designed, and the probes were tested for light-off kinetics and percent signal retention hybridization to complement RNA oligonucleotide targets. Nucleolin probes are shown in SEQ ID NOs:9-17, and tubulin beta-4B probes are shown in SEQ ID NOs:18-21. The probes were labeled with 2-fluoro-acridum ester (2FAE, aka “flasher”) as the reporter.

For light off testing, dilutions of each probe were made and tested at 1:10,000 and 1:5,000. The 1:10 k and 1:5 k dilutions of the probe oligos were created by spiking a 1:100 intermediate dilution into probe reconstitution buffer according to Table 1. 350 μL diH₂O, 100 μL probe (1:10 k and 1:5 k), and 200 μL of oil was spiked into each TTU (ten tube unit) tube, then the TTU was covered with a sealing card and vortexed on a SB100 dry heat bath and vortexer.

TABLE 1

| [Starting] | Final Dilution | μLs Recon | μLs Oligo | Total μL |
|---|---|---|---|---|
| Intermed. 1:100 | 1:5000 | 343.0 | 7.0 | 350.0 |
| Intermed. 1:100 | 1:10000 | 346.5 | 3.5 | 350.0 |

For binding efficiency testing, the average probe concentration used was 0.06 pmol/μL. SEQ ID NO:36 and SEQ ID NO:37 were used as nucleolin probe complement and tubulin beta-4B probe complement, respectively, 100 μL diH₂O, 100 μL probe (targeting 2 million RLU per reaction), 10 μL of complement oligo (200× and 50× dilutions with oligo concentrations as shown in Table 2), and 200 μL of oil was spiked into each TTU tube, then the TTU was covered with a sealing card and run on a SB100 using the following protocol:
1) SB100 ramps up to 62° C. while probe is being added, then place rack on SB100;
2) Vortex—2000 RPM (10 sec);
3) Incubate at 62° C. (20 min);
4) Remove rack and hit next to start RT (room temperature) timer of 5 min;
5) Add Selection Reagent (containing boric acid, sodium hydroxide, and octoxynol; this reagent destroys the label on unbound probe to reduce background noise), then place on SB100;
6) Vortex—1800 RPM (10 sec);
7) Incubate at 62° C. (10 min);
8) Ramp down—62° C. to 23° C.;
9) Incubate at 23° C.; (2 min 30 sec);
10) Remove and read on Leader.

TABLE 2

| | 200X conc. | 50X conc. |
|---|---|---|
| Complement oligo | (pmol/μL) | (pmol/μL) |
| SEQ ID NO: 33 | 12.09 | 3.02 |
| SEQ ID NO: 34 | 12.16 | 3.04 |

Light Off Results. Table 3 shows the average RLU and RLU/μL for each probe (both probe dilutions).

TABLE 3

| Probe | Dilution | Target RLU/μL | Avg RLU | Avg RLU/μL |
|---|---|---|---|---|
| IC probe | 1:10K | 3.82E+04 | 1.66E+06 | 1.66E+04 |
| | 1:5K | 7.64E+04 | 3.43E+06 | 3.43E+04 |
| SEQ ID | 1:10K | 5.04E+04 | 3.01E+06 | 3.01E+04 |
| NO: 9 | 1:5K | 1.01E+05 | 4.84E+06 | 4.84E+04 |
| SEQ ID | 1:10K | 5.51E+04 | 2.93E+06 | 2.93E+04 |
| NO: 10 | 1:5K | 1.10E+05 | 5.36E+06 | 5.36E+04 |
| SEQ ID | 1:10K | 5.16E+04 | 2.87E+06 | 2.87E+04 |
| NO: 11 | 1:5K | 1.03E+05 | 5.16E+06 | 5.16E+04 |
| SEQ ID | 1:10K | 3.60E+04 | 1.96E+06 | 1.96E+04 |
| NO: 12 | 1:5K | 7.21E+04 | 4.21E+06 | 4.21E+04 |
| SEQ ID | 1:10K | 3.88E+04 | 2.17E+06 | 2.17E+04 |
| NO: 13 | 1:5K | 7.76E+04 | 4.23E+06 | 4.23E+04 |
| SEQ ID | 1:10K | 6.01E+04 | 4.21E+06 | 4.21E+04 |
| NO: 14 | 1:5K | 1.20E+05 | 5.92E+06 | 5.92E+04 |
| SEQ ID | 1:10K | 4.36E+04 | 2.15E+06 | 2.15E+04 |
| NO: 15 | 1:5K | 8.72E+04 | 3.46E+06 | 3.46E+04 |
| SEQ ID | 1:10K | 5.97E+04 | 3.40E+06 | 3.40E+04 |
| NO: 16 | 1:5K | 1.19E+05 | 5.69E+06 | 5.69E+04 |
| SEQ ID | 1:10K | 6.05E+04 | 3.53E+06 | 3.53E+04 |
| NO: 17 | 1:5K | 1.21E+05 | 5.39E+06 | 5.39E+04 |
| SEQ ID | 1:10K | 6.02E+04 | 3.08E+06 | 3.08E+04 |
| NO: 18 | 1:5K | 1.20E+05 | 5.24E+06 | 5.24E+04 |
| SEQ ID | 1:10K | 6.48E+04 | 4.44E+06 | 4.44E+04 |
| NO: 19 | 1:5K | 1.30E+05 | 6.31E+06 | 6.31E+04 |
| SEQ ID | 1:10K | 4.79E+04 | 2.88E+06 | 2.88E+04 |
| NO: 20 | 1:5K | 9.57E+04 | 5.13E+06 | 5.13E+04 |
| SEQ ID | 1:10K | 5.57E+04 | 2.81E+06 | 2.81E+04 |
| NO: 21 | 1:5K | 1.11E+05 | 4.59E+06 | 4.59E+04 |

Binding Results. Table 4 shows the average RLU and % retention for each probe, both levels of complement.

TABLE 4

| | Complement | | % |
|---|---|---|---|
| Probe | Dilution | Avg RLU | Retention |
| IC probe | 200x | 1933656 | 96.68% |
| | 50x | 1782874 | 89.14% |
| SEQ ID | 200x | 114110 | 5.71% |
| NO: 9 | 50x | 95527 | 4.78% |
| SEQ ID | 200x | 806593 | 40.33% |
| NO: 10 | 50x | 790158 | 39.51% |
| SEQ ID | 200x | 2482 | 0.12% |
| NO: 11 | 50x | 2303 | 0.12% |
| SEQ ID | 200x | 7135 | 0.36% |
| NO: 12 | 50x | 6147 | 0.31% |
| SEQ ID | 200x | 190473 | 9.52% |
| NO: 13 | 50x | 207422 | 10.37% |
| SEQ ID | 200x | 682861 | 34.14% |
| NO: 14 | 50x | 643768 | 32.19% |
| SEQ ID | 200x | 9224 | 0.46% |
| NO: 15 | 50x | 2009 | 0.10% |
| SEQ ID | 200x | 618180 | 30.91% |
| NO: 16 | 50x | 597370 | 29.87% |
| SEQ ID | 200x | 61281 | 3.06% |
| NO: 17 | 50x | 44681 | 2.23% |
| SEQ ID | 200x | 382578 | 19.13% |
| NO: 18 | 50x | 325389 | 16.27% |
| SEQ ID | 200x | 133747 | 6.69% |
| NO: 19 | 50x | 126671 | 6.33% |
| SEQ ID | 200x | 1244639 | 62.23% |
| NO: 20 | 50x | 987267 | 49.36% |
| SEQ ID | 200x | 1807080 | 90.35% |
| NO: 21 | 50x | 1667103 | 83.36% |

Table 5 shows the interval 25 ratio for the top six probes (three nucleolin and three tubulin beta-4B).

TABLE 5

| Probe | 200X | 50X |
|---|---|---|
| SEQ ID NO: 10 | 1.01 | 0.97 |
| SEQ ID NO: 14 | 1.83 | 1.13 |
| SEQ ID NO: 16 | 0.79 | 1.93 |
| SEQ ID NO: 18 | 1.02 | 1.50 |
| SEQ ID NO: 20 | 0.69 | 0.92 |
| SEQ ID NO: 21 | 0.66 | 0.93 |

Conclusion. Light off testing showed that all probes had strong RLU signal and allowed for value assignment to be performed. For binding testing, the top six probes were picked as having the best % retention and will be tested on the Tigrisn DTS® (Direct Tube Sampling) System with HPV probe oligos added.

### Example 3

The purpose of this study was to see the background and IC flasher signal (from C33A cells for cellularity probes or IC in vitro transcript (IVT) for the IC comparator) of the six best probes chosen from Example 2 in the presence of eight HPV probes.

A probe reagent containing the eight HPV probes and target concentrations listed in Table 6 was prepared.

TABLE 6

| Probe | HPV Target | Conc. (RLU/μL) |
|---|---|---|
| SEQ ID NO: 22 | HPVA1 | 1.50E+05 |
| SEQ ID NO: 23 | HPVA2 | 1.50E+05 |
| SEQ ID NO: 24 | HPV18 | 1.00E+05 |
| SEQ ID NO: 25 | HPV45 | 9.30E+04 |
| SEQ ID NO: 26 | HPV59 | 8.50E+04 |
| SEQ ID NO: 27 | HPVC2 | 1.28E+05 |
| SEQ ID NO: 28 | HPV51 | 1.00E+05 |
| SEQ ID NO: 29 | HPVD | 1.00E+05 |

The probe reagent was then divided in seven with either the internal control (IC) or one of the cellularity control (CC) probes spiked in according to the Table 7 (target RLU for all CC probes was 8.50E+04 RLU/μL).

TABLE 7

| | Probe | 1:100 Dilution | Volume per |
|---|---|---|---|
| Probe | RLU/μL | RLU/μL | probe (μL) |
| IC probe | 3.82E+08 | 3.82E+06 | 24.46 |
| SEQ ID NO: 10 | 5.51E+08 | 5.51E+06 | 16.96 |
| SEQ ID NO: 14 | 3.58E+08 | 3.58E+06 | 26.09 |
| SEQ ID NO: 16 | 5.97E+08 | 5.97E+06 | 15.67 |
| SEQ ID NO: 18 | 2.85E+08 | 2.85E+06 | 32.82 |
| SEQ ID NO: 20 | 4.79E+08 | 4.79E+06 | 19.53 |
| SEQ ID NO: 21 | 5.57E+08 | 5.57E+06 | 16.79 |

C33A cell panel was built to 1e5 cells/mL. IC IVT panel was built to 1425 copies/mL (570 copies/rxn). A condition without IC/CC probe (with only the 8 HPV probes) was used to show how much background signal each CC probe contributes. Tigris DTS reactions (in replicates of 5) were run on SB100 using the following protocol:
1) SB100 ramps up to 62° C. while TCR and sample are added, then place rack on SB100;
2) Vortex—1400 RPM (1 min);
3) Incubate at 62° C. (35 min);
4) Ramp Down—62° C. to 23° C.;
5) Incubate at 23° C. (17 min, 30 sec);
6) Pause for first magwash step;
7) Vortex—1400 RPM (10 sec);
8) Pause for second magwash step;
9) While second magwash step and amp/oil addition is performed, ramp up—23° C. to 62° C.;
10) Vortex—2000 RPM (10 sec);
11) Incubate at 62° C. (10 min);
12) Ramp down—62° C. to 42° C.;
13) Incubate—42° C. (30 sec);
14) Add enzyme;
15) Vortex—700 RPM (5 sec);
16) Incubate at 42° C. (1 hour);
17) Remove rack message flashes, then SB100 lowers from 42° C. to 23° C. and holds temperature until shut down.

Results. Table 8 shows the average RLU for the background (sample transport media (STM)-only panels) and positive panels (IC IVT or C33A cells), along with the % recovery for the positive signals.

TABLE 8

| | Back- | | | | | |
|---|---|---|---|---|---|---|
| | ground | | | | Positive Panels | |
| Probe | Avg | Panel | Conc. | | Avg | % Recovery |
| IC probe | 28682 | IC | 1425 | c/mL | 24420 | 0.29% |
| SEQ ID NO: 10 | 23747 | C33A | 1e5 | c/mL | 185630 | 2.18% |
| SEQ ID NO: 14 | 23137 | C33A | 1e5 | c/mL | 637854 | 7.50% |
| SEQ ID NO: 16 | 59491 | C33A | 1e5 | c/mL | 205016 | 2.41% |
| SEQ ID NO: 18 | 33205 | C33A | 1e5 | c/mL | 98362 | 1.16% |
| SEQ ID NO: 20 | 43835 | C33A | 1e5 | c/mL | 39157 | 0.46% |
| SEQ ID NO: 21 | 30743 | C33A | 1e5 | c/mL | 50393 | 0.59% |

Conclusion. It is unknown why the IC control condition did not produce a positive signal with the IC IVT. It is possible that an incorrect intermediate dilution was used, resulting in a lower concentration than expected.

Out of the cellularity probes, nucleolin probe SEQ ID NO:14 had a low background and the highest average RLU signal in the positive panel and will be used in future testing, and tubulin beta-4B probe SEQ ID NO:18 was chosen as the best of the tubulin beta probes to be used in the next round of testing. These two probes will be put into the probe reconstitution buffer with all eight HPV probes and tested on the Panther® System to determine sensitivity of C33A cells.

### Example 4

The purpose of this study was to run a serial dilution of C33A cells (sensitivity goal is 7.5e2 c/mL in sample transport media (STM; containing LLS, EDTA, and sodium phosphate) with the two cellularity probes chosen in Example 3. Panels with high C33A cells and low concentrations of HeLa (HPV18) and SiHa (HPV16) were also run to see if a high concentration of C33A cells would negatively affect HPV detection.

Two 250 test reaction HPV kits were reconstituted normally apart from target capture reagent (TCR) (no IC added) and probe.

Table 9 shows the build table for the probe reagent containing all eight HPV probes and the two cellularity control probes (one for each kit).

TABLE 9

| Probe | | | Oligo | |
|---|---|---|---|---|
| (SEQ ID NO/Target) | Probe RLU/μL | Target RLU/μL | μL | |
| 22/HPVA1 | 4.97E+08 | 1.50E+05 | 10.99 | |
| 23/HPVA2 | 6.59E+08 | 1.50E+05 | 8.28 | |
| 24/HPV18 | 7.53E+08 | 1.00E+05 | 4.83 | |
| 25/HPV45 | 3.34E+08 | 9.30E+04 | 10.14 | |
| 26/HPV59 | 4.60E+08 | 8.50E+04 | 6.73 | |
| 27/HPVC2 | 6.19E+08 | 1.28E+05 | 7.53 | |
| 28/HPV51 | 5.99E+08 | 1.00E+05 | 6.08 | |
| 29/HPVD | 7.56E+08 | 1.00E+05 | 4.81 | |
| 14/Nucleolin | 358438375 | 1.28E+05 | 12.95 | Kit 1 |
| 18/Tubulin beta-4B | 284927858.3 | 1.28E+05 | 16.29 | Kit 2 |

Serial dilutions of the C33A cells were made according to the Table 10, and intermediate dilutions for SiHa and HcLa were made according to Table 11.

TABLE 10
C33A cells

| [Starting] | [Want] | mLs STM | μLs IVT | Total Vol |
|---|---|---|---|---|
| 1.00E+07 | 2.50E+04 | 57.855 | 145.0 | 58.000 |
| 2.50E+04 | 7.50E+03 | 41.300 | 17,700.00 | 59.000 |
| 7.50E+03 | 2.50E+03 | 37.333 | 18,666.67 | 56.000 |
| 2.50E+03 | 7.50E+02 | 37.100 | 15,900.00 | 53.000 |
| 7.50E+02 | 2.50E+02 | 26.667 | 13,333.33 | 40.000 |

TABLE 11

| | | | | |
|---|---|---|---|---|
| [Starting] | [Want] | mLs STM | μLs IVT | Total Vol |

SiHa Intermediate

| | | | | |
|---|---|---|---|---|
| 1.00E+06 | 1.00E+04 | 0.990 | 10.0 | 1.000 |

HeLa Intermediate

| | | | | |
|---|---|---|---|---|
| 1.00E+06 | 1.00E+04 | 0.495 | 5.0 | 0.500 |
| 1.00E+04 | 1.00E+02 | 0.990 | 10.0 | 1.000 |

The SiHa or HeLa and C33A combined panel were built according to Table 12.

TABLE 12

| | | | | | |
|---|---|---|---|---|---|
| | | | mLs | mLs | Total |
| Organism | [Starting] | [Want] | STM | Lysate | mL |

SiHa3xLoD-C33A 1e5

| | | | | | |
|---|---|---|---|---|---|
| C33A | 1.00E+07 | 2.50E+04 | 39.870 | 0.100 | 40.000 |
| HPV 16 | 1.00E+04 | 7.50E+00 | | 0.030 | |
| (SiHa cells) | | | | | |

SiHa5xLoD-C33A 1e5

| | | | | | |
|---|---|---|---|---|---|
| C33A | 1.00E+07 | 2.50E+04 | 39.850 | 0.100 | 40.000 |
| HPV 16 | 1.00E+04 | 1.25E+01 | | 0.050 | |
| (SiHa cells) | | | | | |

HeLa3xLoD-C33A 1e5

| | | | | | |
|---|---|---|---|---|---|
| C33A | 1.00E+07 | 2.50E+04 | 39.600 | 0.100 | 40.000 |
| HPV 18 | 1.00E+02 | 7.50E−01 | | 0.300 | |
| (HeLa cells) | | | | | |

HeLa5xLoD-C33A 1e5

| | | | | | |
|---|---|---|---|---|---|
| C33A | 1.00E+07 | 2.50E+04 | 39.400 | 0.100 | 40.000 |
| HPV 18 | 1.00E+02 | 1.25E+00 | | 0.500 | |
| (HeLa cells) | | | | | |

The panels listed in Table 13 were tested on the Panthert system with each kit.

TABLE 13

| Panels | Reps |
|---|---|
| Pos/Neg Cals | 6 |
| STM | 60 |
| C33A 2.5e2 | 20 |
| C33A 7.5e2 | 20 |
| C33A 2.5e3 | 20 |
| C33A 7.5e3 | 20 |
| C33A 2.5e4 | 20 |
| SiHa3x-C33A 2.5e4 | 20 |
| SiHa5x-C33A 2.5e4 | 20 |
| HeLa3x-C33A 2.5e4 | 20 |
| HeLa5x-C33A 2.5e4 | 20 |

Results. Table 14 shows average data for the kit with the nucleolin cellularity probe, and Table 15 shows the average data for the kit with the tubulin beta-4B cellularity probe. The HeLa panel positivity is most likely due to the age of the lysate.

TABLE 14
Nucleolin Cellularity Probe Results

| | | | | Positivity | |
|---|---|---|---|---|---|
| | | Cellularity | HPV | S/CO | % |
| Panel | n | Avg | Avg | Avg | Positivity |
| Neg STM | 60 | 14231 | 43280 | 0.05 | 0.0% |
| C33A 2.5e2 | 20 | 1177460 | 4222 | 0.00 | 0.0% |
| C33A 7.5e2 | 20 | 1101352 | 0 | 0.00 | 0.0% |
| C33A 2.5e3 | 20 | 1059237 | 1976 | 0.00 | 0.0% |
| C33A 7.5e3 | 20 | 1024270 | 25286 | 0.03 | 0.0% |
| C33A 2.5e4 | 20 | 972316 | 8201 | 0.01 | 0.0% |
| SiHa3x-C33A 2.5e4 | 20 | 714234 | 7145164 | 8.25 | 100.0% |
| SiHa5x-C33A 2.5e4 | 20 | 702851 | 7996012 | 9.23 | 100.0% |
| HeLa3x-C33A 2.5e4 | 20 | 939369 | 1017379 | 1.17 | 40.0% |
| HeLa5x-C33A 2.5e4 | 18 | 908366 | 1624039 | 1.87 | 66.7% |

TABLE 15
Tubulin beta-4B Cellularity Probe Results

| | | | | Positivity | |
|---|---|---|---|---|---|
| | | Cellularity | HPV | S/CO | % |
| Panel | n | Avg | Avg | Avg | Positivity |
| Neg STM | 60 | 23384 | 57588 | 0.07 | 0.0% |
| C33A 2.5e2 | 20 | 20972 | 49865 | 0.06 | 0.0% |
| C33A 7.5e2 | 20 | 20595 | 49051 | 0.06 | 0.0% |
| C33A 2.5e3 | 20 | 23543 | 56534 | 0.07 | 0.0% |
| C33A 7.5e3 | 20 | 28977 | 71251 | 0.08 | 0.0% |
| C33A 2.5e4 | 20 | 26124 | 60265 | 0.07 | 0.0% |
| SiHa3x-C33A 2.5e4 | 20 | 0 | 6242873 | 7.43 | 100.0% |
| SiHa5x-C33A 2.5e4 | 20 | 0 | 7217407 | 8.58 | 100.0% |
| HeLa3x-C33A 2.5e4 | 20 | 9012 | 1233578 | 1.47 | 80.0% |
| HeLa5x-C33A 2.5e4 | 18 | 6534 | 1745022 | 2.08 | 88.9% |

Conclusion. The high positive signal of the HPV (SiHa & HeLa) panels was due to the target RLU/μL used as the target and the dilution of that concentration into the reconstitution buffer was not accounted for, resulting in target RLU being around 1 log higher than it should have been.

The nucleolin probe performed substantially better than the tubulin beta-4B probe as a cellularity control.

### Example 5

The purpose of this study was to adjust RLU/μL input of the nucleolin cellularity probe to produce a flasher IC signal around 250 k RLU.

HPV 100 test reaction kit reagents were reconstituted normally apart from TCR (no IC added) and probe.

Probe reconstitution buffer was spiked with HPV probes and nucleolin cellularity probe according to the Table 16.

TABLE 16

| Probe | Probe RLU/μL | | Target RLU/μL | | | |
|---|---|---|---|---|---|---|
| (SEQ ID NO/ | | 1:00 | Lyo | Adjusted* | Oligo | |
| Target) | Oligo | Dilution | RLU/μL | RLU/μL | μL | |
| 22/HPVA1 | 4.97E+08 | 4.97E+06 | 1.50E+05 | 1.53E+04 | 48.3 | |
| 23/HPVA2 | 6.59E+08 | 6.59E+06 | 1.50E+05 | 1.53E+04 | 36.4 | |
| 24/HPV18 | 7.53E+08 | 7.53E+06 | 1.00E+05 | 1.02E+04 | 21.2 | |
| 25/HPV45 | 3.34E+08 | 3.34E+06 | 9.30E+04 | 9.48E+03 | 44.6 | |
| 26/HPV59 | 4.60E+08 | 4.60E+06 | 8.50E+04 | 8.66E+03 | 29.6 | |
| 27/HPVC2 | 6.19E+08 | 6.19E+06 | 1.28E+05 | 1.30E+04 | 33.1 | |
| 28/HPV51 | 5.99E+08 | 5.99E+06 | 1.00E+05 | 1.02E+04 | 26.7 | |
| 29/HPVD | 7.56E+08 | 7.56E+06 | 1.00E+05 | 1.02E+04 | 21.2 | |
| 14/Nucleolin | 358438375 | 3.58E+06 | 1.28E+05 | 1.30E+04 | 56.9 | Kit 1 |
| 14/Nucleolin | 358438375 | 3.58E+06 | 8.50E+04 | 8.66E+03 | 37.9 | Kit 2 |
| 14/Nucleolin | 358438375 | 3.58E+06 | 2.46E+05 | 2.51E+04 | 109.8 | Kit 3 |

*The adjusted RLU/μL was found by multiplying the lyo RLU/μL by the ratio of lyo volume divided by recon volume (0.102 for the 100tk).

C33A cells were used to build the panels listed in Table 17 and Table 18.

TABLE 17
C33A cells

| | | mLs | μLs | Total |
|---|---|---|---|---|
| [Starting] | [Want] | STM | IVT | Vol |
| 1.00E+07 | 2.50E+04 | 25.935 | 65.0 | 26.000 |
| 2.50E+04 | 7.50E+02 | 33.465 | 1035.0 | 34.500 |
| 7.50E+02 | 2.50E+02 | 18.667 | 9333.3 | 28.000 |
| 7.50E+02 | 7.50E+01 | 22.500 | 2500.0 | 25.000 |

TABLE 18

| | | | | |
|---|---|---|---|---|
| | | mLs | μLs | Total |
| [Starting] | [Want] | STM | IVT | Vol |

SiHa Intermediate

| | | | | |
|---|---|---|---|---|
| 1.00E+06 | 1.00E+04 | 0.990 | 10.0 | 1.000 |

HeLa Intermediate

| | | | | |
|---|---|---|---|---|
| 1.00E+06 | 1.00E+04 | 0.498 | 2.5 | 0.500 |
| 1.00E+04 | 1.00E+02 | 0.990 | 10.0 | 1.000 |

The SiHa or HeLa and C33A combined panel were built according to Table 19.

TABLE 19

| | | | | | |
|---|---|---|---|---|---|
| | | | mLs | mLs | Total |
| Organism | [Starting] | [Want] | STM | Lysate | mL |

SiHa3xLoD-C33A 1e5

| | | | | | |
|---|---|---|---|---|---|
| C33A | 1.00E+07 | 2.50E+04 | 24.919 | 0.063 | 25.000 |
| HPV 16 | 1.00E+04 | 7.50E+00 | | 0.019 | |
| (SiHa cells) | | | | | |

SiHa5xLoD-C33A 1e5

| | | | | | |
|---|---|---|---|---|---|
| C33A | 1.00E+07 | 2.50E+04 | 24.906 | 0.063 | 25.000 |
| HPV 16 | 1.00E+04 | 1.25E+01 | | 0.031 | |
| (SiHa cells) | | | | | |

SiHa100x-C33A 7.5e2

| | | | | | |
|---|---|---|---|---|---|
| C33A | 1.00E+07 | 7.50E+02 | 24.992 | 0.002 | 25.000 |
| HPV 18 | 1.00E+06 | 2.50E+02 | | 0.006 | |
| (HeLa cells) | | | | | |

HeLa3xLoD-C33A 1e5

| | | | | | |
|---|---|---|---|---|---|
| C33A | 1.00E+07 | 2.50E+04 | 24.750 | 0.063 | 25.000 |
| HPV 16 | 1.00E+02 | 7.50E−01 | | 0.188 | |
| (SiHa cells) | | | | | |

HeLa5xLoD-C33A 1e5

| | | | | | |
|---|---|---|---|---|---|
| C33A | 1.00E+07 | 2.50E+04 | 24.625 | 0.063 | 25.000 |
| HPV 16 | 1.00E+02 | 1.25E+00 | | 0.313 | |
| (SiHa cells) | | | | | |

HeLa100x-C33A 7.5e2

| | | | | | |
|---|---|---|---|---|---|
| C33A | 1.00E+07 | 7.50E+02 | 24.936 | 0.002 | 25.000 |
| HPV 18 | 1.00E+04 | 2.50E+01 | | 0.063 | |
| (HeLa cells) | | | | | |

The panels listed in Table 20 were tested on the Panther® system.

TABLE 20

| Panels | Reps |
|---|---|
| Pos/Neg Cals | 6 |
| STM | 8 |
| C33A 7.5e1 | 8 |
| C33A 2.5e2 | 8 |
| C33A 7.5e2 | 8 |
| C33A 2.5e4 | 8 |
| SiHa100x-C33A 7.5e2 | 8 |
| HeLa100x-C33A 7.5e2 | 8 |
| SiHa3x-C33A 2.5e4 | 8 |
| SiHa5x-C33A 2.5e4 | 8 |
| HeLa3x-C33A 2.5e4 | 8 |
| HeLa5x-C33A 2.5e4 | 8 |

The cellularity output was lower than desired in kits 1 and 2, so a second round of testing was performed. For round 2, the concentration was increased to that listed in kit 3 (see Table 16) and the same panel setup was used with the only the following change: C33A cells were pipetted into the HPV negative calibrator according to Table 21.

TABLE 21
C33A cells

| | | mLs | μLs | Total |
|---|---|---|---|---|
| [Starting] | [Want] | STM | IVT | Vol |
| 1.00E+07 | 5.00E+03 | 2.699 | 1.4 | 2.7 |

Due to a possible mis-spike/contamination affecting the results of kit 3, a new kit was built and tested in a third round. For round 3, C33A cells were pipetted into both the HPV negative and positive calibrators, following the previous table (Table 21).

Additional SiHa and HeLa high concentration panels were built according to Table 22.

TABLE 22

| | | | | |
|---|---|---|---|---|
| | | mLs | μLs | Total |
| [Starting] | [Want] | STM | IVT | Vol |

SiHa High Panel

| | | | | |
|---|---|---|---|---|
| 1.00E+04 | 2.50E+02 | 4.875 | 125.0 | 5.000 |

HeLa High Panel

| | | | | |
|---|---|---|---|---|
| 1.00E+04 | 2.50E+01 | 4.988 | 12.5 | 5.000 |

For round 3, the panels listed in Table 20 were tested on the Panther® system.

Results. Results are shown in Tables 23-26. Table 23 shows the averages for the 1.3e4 RLU/μL CC probe kit.

TABLE 23

| | | | | Positivity | |
|---|---|---|---|---|---|
| | | Cellularity | HPV | S/CO | % |
| Panel | n | Avg | Avg | Avg | Positivity |
| STM | 8 | 1217 | 4025 | 0.04 | 0.0% |
| C33A 7.5e1 | 8 | 116582 | 800 | 0.01 | 0.0% |
| C33A 2.5e2 | 8 | 125559 | 0 | 0.00 | 0.0% |
| C33A 7.5e2 | 8 | 115985 | 549 | 0.01 | 0.0% |
| C33A 2.5e4 | 8 | 99040 | 3233 | 0.03 | 0.0% |
| SiHa3x-C33A 2.5e4 | 8 | 55951 | 828315 | 8.63 | 100.0% |
| SiHa5x-C33A 2.5e4 | 8 | 44679 | 943465 | 9.84 | 100.0% |
| SiHa100x-C33A 7.5e2 | 8 | 40341 | 1098708 | 11.45 | 100.0% |
| HeLa3x-C33A 2.5e4 | 8 | 49856 | 797722 | 8.32 | 100.0% |
| HeLa5x-C33A 2.5e4 | 8 | 53768 | 789061 | 8.23 | 100.0% |
| HeLa100x-C33A 7.5e2 | 8 | 49439 | 965086 | 10.06 | 100.0% |

Table 24 shows the averages for the 8.7e3 RLU/μL CC probe kit.

TABLE 24

| | | | | Positivity | |
|---|---|---|---|---|---|
| | | Cellularity | HPV | S/CO | % |
| Panel | n | Avg | Avg | Avg | Positivity |
| STM | 8 | 1060 | 4286 | 0.04 | 0.0% |
| C33A 7.5e1 | 8 | 80169 | 2980 | 0.03 | 0.0% |
| C33A 2.5e2 | 8 | 80510 | 474 | 0.00 | 0.0% |
| C33A 7.5e2 | 8 | 76910 | 1994 | 0.02 | 0.0% |
| C33A 2.5e4 | 8 | 64558 | 909 | 0.01 | 0.0% |
| SiHa100x-C33A 7.5e2 | 8 | 31618 | 738116 | 7.74 | 100.0% |
| HeLa100x-C33A 7.5e2 | 8 | 14961 | 954642 | 10.01 | 100.0% |
| SiHa3x-C33A 2.5e4 | 8 | 11988 | 1090663 | 11.43 | 100.0% |
| SiHa5x-C33A 2.5e4 | 8 | 20792 | 788828 | 8.27 | 100.0% |
| HeLa3x-C33A 2.5e4 | 8 | 14536 | 796501 | 8.35 | 100.0% |
| HcLa5x-C33A 2.5c4 | 8 | 19730 | 959899 | 10.06 | 100.0% |

Table 25 shows the averages for the 2.5e4 RLU/μL CC probe kit. PG-5T

TABLE 25

| | | | | Positivity | |
|---|---|---|---|---|---|
| | | Cellularity | HPV | S/CO | % |
| Panel | n | Avg | Avg | Avg | Positivity |
| STM | 8 | 3737 | 6781 | 0.09 | 0.0% |
| C33A 7.5e1 | 8 | 209073 | 0 | 0.00 | 0.0% |
| C33A 2.5e2 | 8 | 224980 | 0 | 0.00 | 0.0% |
| C33A 7.5e2 | 8 | 214409 | 0 | 0.00 | 0.0% |
| C33A 2.5e4 | 8 | 179178 | 0 | 0.00 | 0.0% |
| SiHa100x-C33A 7.5e2 | 8 | 311492 | 581240 | 7.93 | 100.0% |
| HeLa100x-C33A 7.5e2 | 8 | 322934 | 728897 | 9.95 | 100.0% |
| SiHa3x-C33A 2.5e4 | 8 | 388289 | 851329 | 11.62 | 100.0% |
| SiHa5x-C33A 2.5e4 | 8 | 300403 | 634972 | 8.66 | 100.0% |
| HeLa3x-C33A 2.5e4 | 8 | 302890 | 629624 | 8.59 | 100.0% |
| HeLa5x-C33A 2.5e4 | 8 | 375342 | 752184 | 10.26 | 100.0% |

Table 26 shows the averages for the 2.5e4 RLU/μL CC probe kit (round 3). PG-6T,

TABLE 26

| | | | | Positivity | |
|---|---|---|---|---|---|
| | | Cellularity | HPV | S/CO | % |
| Panel | n | Avg | Avg | Avg | Positivity |
| STM | 7 | 2857 | 6705 | 0.07 | 0.0% |
| C33A 75 c/ml | 7 | 206398 | 0 | 0 | 0.0% |
| C33A 250 c/ml | 7 | 236558 | 0 | 0 | 0.0% |
| C33A 750 c/ml | 7 | 223378 | 0 | 0 | 0.0% |
| C33A 25,000 c/ml | 7 | 178792 | 0 | 0 | 0.0% |
| SiHa3x-C33A 2.5e4 | 7 | 195533 | 542753 | 5.95 | 100.0% |
| SiHa5x-C33A 2.5e4 | 7 | 171730 | 901079 | 9.88 | 100.0% |
| SiHa100x-C33A 7.5e2 | 7 | 199611 | 1030257 | 11.30 | 100.0% |
| HeLa3x-C33A 2.5e4 | 7 | 165559 | 745754 | 8.18 | 100.0% |
| HeLa5x-C33A 2.5e4 | 7 | 184522 | 746941 | 8.19 | 100.0% |
| HeLa100x-C33A 7.5e2 | 7 | 183825 | 905497 | 9.93 | 100.0% |
| SiHa100x (250) | 7 | 199176 | 1022736 | 11.21 | 100.0% |
| HeLa100x (25) | 7 | 228182 | 850724 | 9.33 | 100.0% |
| Pos Cal (No C33A) | 3 | 43923 | 997847 | 10.94 | 100.0% |

Conclusion/Discussion. The lower two concentrations had signal that was lower than desired. Increasing the cellularity probe concentration to 2.5e4 RLU/L resulted in RLU output around 220 k RLU, which was considered acceptable.

A second kit of the 2.5e4 RLU/μL was made and run since there were some oddities in the first kit tested (C33A cells were not added to the positive calibrator but it had CC signal equal to the negative calibrator, which contained C33A cells and HPV signal was much lower than expected). This was most likely due to a kit mis-spike, as rebuilding the kit eliminated the strange results.

TABLE 27
Sequences

| SEQ | | |
|---|---|---|
| ID | | |
| NO | Sequence (5′ to 3′) | Description |
| 1 | GenBank Accession | Nucleolin gene |
| | No. NM_005381.3 | |
| 2 | GACAGCTCAGAGGAGGAGGATG | Amplification oligomer |
| 3 | GTCGTCGACAAATACCTGTGTGTG | Amplification oligomer |
| | ATAGGAGAGGGATATCACTCAG | |
| | CATAATTTAA | |
| 4 | GGAGAGGGATATCACTCAGCATAATTTAA | THS† of SEQ ID NO: 3 |
| 5 | GAGGAUGAAGAUGAAAUUG | Nucleolin detection probe |
| 6 | GGAUGAAGAUGAAAUUGAAC | Nucleolin detection probe |
| 7 | GAUGAAGAUGAAAUUGAAC | Nucleolin detection probe |
| 8 | AGCAAAUGCUUAAUGUCC | Tubulin beta-4B detection |
| | | probe |
| 9 | GAGGAUG*AAGAUGAAAUUG | Nucleolin detection probe |
| 10 | GAGGAUGA*AGAUGAAAUUG | Nucleolin detection probe |
| 11 | GAGGAUGAAG*AUGAAAUUG | Nucleolin detection probe |
| 12 | GGAUGAAG*AUGAAAUUGAAC | Nucleolin detection probe |
| 13 | GGAUGAAGA*UGAAAUUGAAC | Nucleolin detection probe |
| 14 | GGAUGAAGAU*GAAAUUGAAC | Nucleolin detection probe |
| 15 | GAUGAAG*AUGAAAUUGAAC | Nucleolin detection probe |
| 16 | GAUGAAGAU*GAAAUUGAAC | Nucleolin detection probe |
| 17 | GAUGAAGAUG*AAAUUGAAC | Nucleolin detection probe |
| 18 | AGCAAAU*GCUUAAUGUCC | Tubulin beta-4B detection |
| | | probe |
| 19 | AGCAAAUG*CUUAAUGUCC | Tubulin beta-4B detection |
| | | probe |
| 20 | AGCAAAUGCU*UAAUGUCC | Tubulin beta-4B detection |
| | | probe |
| 21 | AGCAAAUGCUU*AAUGUCC | Tubulin beta-4B detection |
| | | probe |
| 22 | CAGCUGGACAAGCAGAACCGGAC | HPVAl detection probe |
| 23 | GGCCAGAUGGACAAGCACAAC | HPVA2 detection probe |
| 24 | GUAGUAGAAAGCUCAGCAGACG | HPV18 detection probe |
| | ACC | |
| 25 | GUAGAGAGCUCGGCAGAGGAC | HPV45 detection probe |
| 26 | GUAGUAGAAACCUCGCAAGACGG | HPV59 detection probe |
| 27 | CCGACCAUGCAGUUAAUCACC | HPVC2 detection probe |
| 28 | GCGUGACCAGCUACCAGAAAG | HPV51 detection probe |
| 29 | CACGUACCUUGUUGUGAGUG | HPVD detection probe |
| 30 | GACAGTGATGAAGAGGAGGATGAT | Nucleolin amplification |
| | GACAGTGAGGAGGATGAGGAGGAT | oligomer binding region |
| | GACGAGGACGAGGATGAG | |
| 31 | CAGCAGCGATGAAAGCAGCAGCTG | Nucleolin amplification |
| | CTGCCCCT | oligomer binding region |
| 32 | GACAGTGATGAAGAGGAGGATGAT | Nucleolin target region |
| | GACAGTGAGGAGGATGAGGAGGAT | |
| | GACGAGGACGAGGATGAGGATGAA | |
| | GATGAAATTGAACCAGCAGCGATG | |
| | AAAGCAGCAGCTGCTGCCCCT | |
| 36 | GUUCAAUUUCAUCUUCAUCCUCAU | Nucleolin detection probe |
| | CCUCGUCCUCGUCAU | complement, methoxy |
| | | backbone |
| 37 | UUUUUGUUUUGGACAUUAAGCAU | Tubulin beta-4B detection |
| | UUGCU | probe complement, |
| | | methoxy backbone |

†THS = Target-hybridizing sequence
*Acridinium Ester (AE) label

From the foregoing, it will be appreciated that, although specific embodiments of the invention have been described herein for purposes of illustration, various modifications may be made without deviating from the spirit and scope of the invention. Accordingly, the invention is not limited except as by the appended claims. All publications, patents, and patent applications cited herein are hereby incorporated by reference in their entireties for all purposes.

1. A method for performing an amplification assay to determine the presence or absence of a human papillomavirus (HPV) target nucleic acid in a biological sample, wherein the HPV target nucleic acid is selected from the group consisting of HPV types 16 and 59, the method comprising:
(a) providing a biological sample suspected of containing human eukaryotic cells that may comprise the target nucleic acid;
(b) contacting the sample with at least two control amplification oligomers and at least two target-specific amplification oligomers, wherein the at least two control amplification oligomers are capable of amplifying a target region of a nucleolin control nucleic acid, wherein the at least two target-specific amplification oligomers are capable of amplifying a target region of the HPV target nucleic acid, and wherein
(i) the first control amplification oligomer comprises a first target-hybridizing sequence as shown in SEQ ID NO:2, and/or
(ii) the second control amplification oligomer comprises a second target-hybridizing sequence as shown in SEQ ID NO:4;
(c) performing an in vitro nucleic acid amplification reaction, wherein any nucleolin control nucleic acid present in the sample is used as a template for generating a control amplification product, and wherein any HPV target nucleic acid present in the sample is used as a template for generating a target amplification product; and
(d) detecting the presence or absence of each of the control amplification product and the target amplification product, wherein if the control amplification product is present, then the presence of the eukaryotic cells in the sample is assured and the presence or absence of the target amplification product determines, respectively, the presence or absence of the target nucleic acid in the sample.

2-5. (canceled)

6. The method of claim 1, wherein the sample is a tissue swab,
optionally wherein the tissue swab is a cervical swab, a vaginal swab, a nasal swab, a buccal swab, a throat swab, an anal swab, a urethral/penile swab, or a swab from an open wound/sore/laceration.

7. (canceled)

8. The method of claim 1, wherein the target nucleic acid is an RNA.

9-12. (canceled)

13. The method of claim 1, wherein the amplification assay is a multiplex amplification assay comprising detection of two or more types of HPV.

14. The method of claim 13, wherein the two or more types of HPV comprise two or more of HPV types 16, 18, 31, 33, 35, 39, 45, 51, 52, 56, 58, 59, 66, and 68.

15. The method of claim 14, wherein the two or more types of HPV comprise each of HPV type 16 (HPV16) and HPV type 59 (HPV59).

16. The method of claim 15, wherein the at least two target-specific amplification oligomers comprise
(i) an HPV16-specific amplification oligomer comprising a target-hybridizing as shown in SEQ ID NO:2, and
(ii) an HPV59-specific amplification oligomer comprising a target-hybridizing as shown in SEQ ID NO:4.

17. (canceled)

18. The method of claim 1, wherein generating and detecting the control amplification product as defined in steps (b)-(d) is performed in a multiplex format with generating and detecting the target amplification product.

19. (canceled)

20. The method of claim 1, wherein the detecting step (d) comprises contacting the in vitro nucleic acid amplification reaction with
(i) at least one control detection probe oligomer configured to specifically hybridize to the control amplification product under conditions whereby the presence or absence of the control amplification product is determined, and
(ii) at least one target-specific detection probe oligomer configured to specifically hybridize to the target amplification product under conditions whereby the presence or absence of the target amplification product is determined,
thereby detecting the presence or absence of each of the control amplification product and the target amplification product.

21. The method of claim 20, wherein the at least one control detection probe oligomer comprises a target-hybridizing sequence selected from the group consisting of SEQ ID NO:5, SEQ ID NO:6, and SEQ ID NO:7.

22-27. (canceled)

28. The method of claim 20, wherein each of the at least one control detection probe oligomer and the at least one target-specific detection probe oligomer comprises a detectable label.

29. The method of claim 28, wherein the detectable label is a chemiluminescent or fluorescent label.

30. The method of claim 29, wherein the detectable label is a chemiluminescent label.

31. The method of claim 30, wherein the chemiluminescent label is an acridinium ester (AE) compound.

32. The method of claim 31, wherein the at least one control detection probe oligomer comprises a target-hybridizing sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

33. The method of claim 32, wherein the at least one control detection probe oligomer comprises a target-hybridizing sequence selected from the group consisting of SEQ ID NO:10, SEQ ID NO:14, and SEQ ID NO:16.

34. (canceled)

35. (canceled)

36. The method of claim 1, wherein the in vitro nucleic acid amplification reaction is an isothermal amplification reaction.

37. The method of claim 36, wherein the isothermal amplification reaction is a transcription-mediated amplification reaction.

38. A composition or kit for performing an amplification assay to determine the presence or absence of a human papillomavirus (HPV) target nucleic acid in a sample, wherein the HPV target nucleic acid is selected from the group consisting of HPV types 16 and 59, the composition or kit comprising:
at least two control amplification oligomers and at least two target-specific amplification oligomers, wherein the at least two control amplification oligomers are capable of amplifying a target region of a nucleolin control nucleic acid, wherein the at least two target-specific amplification oligomers are capable of amplifying a target region of the HPV target nucleic acid, and wherein
(i) the first control amplification oligomer comprises a first target-hybridizing sequence as shown in SEQ ID NO:2, and/or
(ii) the second control amplification oligomer comprises a second target-hybridizing sequence as shown in SEQ ID NO:4.

39-81. (canceled)

82. A detection probe oligomer comprising:
a nucleolin-specific detection probe target-hybridizing sequence that is from about 15 to about 35 nucleotides in length and is configured to hybridize to a target sequence contained within a target region of human nucleolin nucleic acid corresponding to a region of SEQ ID NO:1 from about nucleotide position 585 to about nucleotide position 677.

83-116. (canceled)
